(19) United States
(12) Patent Application Publication
(10) Pub. No.: US 2025/0195196 A1
(43) Pub. Date: Jun. 19, 2025

(54) PHYSIOLOGICALLY AND MECHANICALLY BIOCOMPATIBLE ARTIFICIAL PANCREAS AND PHYSICALLY CROSSLINKED POLYVINYL ALCOHOL HYDROGELS AND SOLIDS FROM WHICH THE BIOARTIFICIAL PANCREAS IS MANUFACTURED

(71) Applicant: Nedeljko Vladimira Gvozdic, Fort Myers, FL (US)

(72) Inventor: Nedeljko Vladimira Gvozdic, Fort Myers, FL (US)

(21) Appl. No.: 18/982,079

(22) Filed: Dec. 16, 2024

Related U.S. Application Data
(60) Provisional application No. 63/611,738, filed on Dec. 18, 2023.

(51) Int. Cl. A61F 2/02; A61L 27/16; A61L 27/36; A61L 27/38; A61L 27/52; A61L 27/54; B33Y 80/00; C08L 29/04

(52) U.S. Cl. CPC A61F 2/022; A61L 27/16; A61L 27/3625; A61L 27/3804; A61L 27/52; A61L 27/54; B33Y 80/00; C08L 29/04; A61F 2220/0075; A61F 2250/0067; A61L 2300/64

(57) ABSTRACT

The development of devices for a functional cure for diabetes by mimicking native pancreatic tissue for production of all pancreatic hormones. A physiologically and mechanically biocompatible artificial pancreas, wherein the pancreas comprises a combination of a PVA hydrogel combined with cells selected from the group consisting of islets; clusters of islets; Beta cells, and combinations of islets and Beta cells. The bioartificial pancreas contains vascular grafts therein or thereon a wall of the pancreas so that said grafts are an integral part of the wall of the bioartificial pancreas. The grafts can be anasmotized to living blood vessels in the body.

### CROSS-REFERENCE TO RELATED APPLICATIONS

This application is a utility application claiming the benefit and priority of U.S. Provisional application Ser. No. 63/611,738, filed Dec. 18, 2023.

### STATEMENT REGARDING FEDERALLY SPONSORED RESEARCH OR DEVELOPMENT

Not Applicable.

### REFERENCE TO SEQUENCE LISTING, A TABLE, OR A COMPUTER PROGRAM LISTING COMPACT DISC APPENDIX

Not Applicable.

### BACKGROUND OF THE INVENTION

What is disclosed herein is the development of devices for use in functional cure for diabetes by mimicking native pancreatic tissue for production of pancreatic hormones.

A literature search revealed a large number of publications that have attempted to develop such a functional cure but without apparent prospects for success in the foreseeable future.

The long history of diabetes mellitus therapy shows steady progress of the development of “mechanical” devices that can deliver insulin to mitigate some of the potentially serious issues related to diabetes mellitus caused by the loss of beta cell activity. There is an obvious continuous incremental improvement in automation of monitoring blood glucose levels (BGL) and, timely injection of required insulin to a patients' tissue to “mechanically” regulate blood glucose levels (GBL).

The last two decades have marked accelerated improvements in the sophistication of automated electronic monitoring and automated activation of insulin delivery pumps. Focus on the improvement of mechanical delivery devices is taking place in spite of the many drawbacks of the “mechanical” approach to deal with diabetes, as one would expect when one tries to use mechanical devices to mimic native cellular processes.

It seems that among important reasons for efforts to improve “mechanical” devices dealing with diabetes mellitus, there are tenacious hurdles that are still preventing development of alternative paths that would deal with more natural therapies for diabetes mellitus. The most prominent reason is a failure to develop a bioartificial pancreas (BAP), a medical device that would house islets or at least beta cells within non-immunogenic protective devices made in the form of a pouch that can be surgically implanted into well-vascularized tissue.

One can trace back to the mid-1980s' as a time of the onset of concerted efforts to develop a BAP, wherein pancreatic cells would reside within an environment conducive to establishment of the cell's hemostasis, that is, conditions that would allow cells to function within immune-isolating space as if they reside in a native pancreas.

Only under such conditions will surgical implantation of immune-isolated pancreatic cells provide a functional cure for diabetes mellitus that are caused by the loss of beta cell function.

Since the mid-1980s' myriad variations of designs of BAP devices have been reported using a wide range of biomaterials and very creative engineering designs for the BAP, which were combined with quite astute surgical protocols for implantation of the devices into different tissues in an attempt to provide an environment that could closely mimic native pancreatic conditions.

At an early stage of the development of a BAP there was some excitement about the prospects of placing BAP into a bloodstream, the most desirable place. However, that excitement quickly subsided because it was soon realized that such a route of development faced major intractable hurdles that none of the biomaterials to date could overcome.

This is actually the most difficult case of BAP design because the design of such a BAP requires availability of biomaterials that can concurrently meet the following requirements: (a) provide immune-isolation of the pancreatic cells, (b) provide free exchange of molecular species through the membrane walls and (c) provide surfaces that are not thrombogenic, not only at the site of implantation but also downstream of the implantation.

Lack of adequate biomaterial has led to an abandonment of this route during the last 20 years. Generally, one reason for abandonment is the realization that placement of a BAP into a blood stream is far from being feasible in the near future based on the state of current biotechnology developments and the knowledge base required to deal with imposed hurdles.

To date, none of the numerous efforts has led to the development of a BAP that can provide a functional cure for diabetes mellitus. Currently, a widely shared belief is that the present state of the art of development of BAP shows no prospects for development of a functional cure for diabetes, using surgically implanted immune-isolated pancreatic cells.

Biocompatible artificial pancreas dealt with in the prior art and based on the use of various hydrogels include U.S. Pat. Nos. 10,835,486, 10,786,446, and U.S. Publication 2021/0069100 that disclose methods of making multi-layer hydrogel capsules using alginate as the hydrogel matrix.

U.S. Pat. No. 12,115,332 discloses a method of encapsulating pancreatic cells and agents using polysulfone, polyethersulfone, cellulose, nitrate, and polyethylene, among others.

U.S. Patent Publication 2022/0184280 discloses therapeutic hydrogel-based devices that are made from agarose hydrogels.

U.S. Pat. No. 10,835,609 discloses therapeutic hydrogel-based devices using methacrylate copolymers and peptide linkers.

U.S. Publication 2017/0157294 discloses an optimization process for macro encapsulation of islets in alginate-based hydrogel.

U.S. Pat. No. 10,934,529 discloses hydrogels using crosslinked hydrophilic polymers such as polyethylene glycol.

U.S. Pat. Nos. 11,058,795, and 11,058,795, and U.S. Patent Publication 2019/0015547 disclose the use of ethylene-vinyl alcohol copolymers with sulfone polymers, polyacrylonitrile, cellulose, polyamide and polycarbonate, in various ratios.

Japanese Unexamined Patent Application Publication 2004-275718 discloses a bioartificial pancreas to be worn externally on a body. The hollow fibers are composed of ethylene-vinyl alcohol base copolymers.

Japanese Unexamined Patent Application Publication 2001-314736 discloses hollow fiber membranes composed of a polymer semi-permeable membrane having high permeability.

U.S. Patent Publication 2020/0231960 that published on Jul. 23, 2020, to Oharuda, et al. discloses a cell or tissue embedding device highly capable of supplying a physiologically active substance, by curbing the reduction of living cells or living tissue, in a process of preparing a polyvinyl alcohol gel containing the living cells or living tissue. The aqueous gel forms an immuno-isolation layer of a cell or tissue embedding device that has, as a component, a polyvinyl alcohol resin having a syndiotacticity of 32 to 40% in triad.

The patentees claim the use of polyvinyl alcohol having a syndiotacticity of 32 to 40%. Syndiotacticity is important. In the instant invention, a high degree of syndiotacticity is preferred, ideally 100%. One needs to have a high degree of syndiotacticity as possible, ideally 100% and 32 to 40% will play no role in the use of the instant device.

This patent claims a degree of hydrolysis of 88.9% to 90% but one should keep in mind that that polyvinyl alcohol is very different from what is used in the instant invention in view of the fact that they start with poly(vinyl pivalate) (PVP) and the instant invention starts with poly(vinyl acetate). The patentees end up with a different PVA polymer if they do not hydrolyze PVP to 100% which is difficult to achieve with PVP. Their polymer would require even a higher degree of hydrolysis than the instant polymer due to area functional groups of the PVP polymer that readily disrupt immobilized water layers

In addition, their polymers require support, because their polymer is weak and needs the support. Thus, they use PE, PP, and Teflon® as support materials, in that, the instant invention polymers do not require. Also, their support creates bio-compatibility issues in the body.

U.S. Patent Publications 2016/0206741, 2018/0256,724 and U.S. Pat. No. 9,937,256 disclose methacrylate copolymers cross-linked with cleavable peptide linkers.

U.S. Patent Publication 2022/0071920 discloses a method of making semi-permeable membranes comprising at least two fibrous layers using polyurethane polymer fibers.

U.S. Patent Publication 2020/0289709 discloses the use of 3-D printing based on gelated matrices using hyaluronic acid, agarose, alginate, and poly(ethylene-glycol) polymers.

U.S. Pat. No. 10,730,983 discloses zwitterionic polymers or biocompatible polymers for cell encapsulation and coating of a device.

U.S. Patent Publication 2022/0002667 discloses compositions and methods for generating one or more scaffolds. The aqueous process consists of elastin, alginate, PEO, collagen and chitosan.

U.S. Patent Publication 2024/0139379 discloses compositions and methods for using bio-scaffolds using cryoelectrospinning. They use elastin, alginate, PEO, collagen, and chitosan.

None of the prior art discussed Supra discloses the instant invention or makes it obvious.

### BRIEF SUMMARY OF THE INVENTION

Thus, what is disclosed and claimed in the instant invention, in one embodiment, is a physically crosslinked polyvinyl alcohol hydrogel prepared using mixed solvents. The hydrogel contains continuous, uninterrupted immobilized water. The hydrogels have at least 88% by weight water content or higher and a syndiotacticity of fifty percent or greater.

The hydrogels of this invention are unique. They must have a syndiotacticity of fifty percent or greater, have a molecular weight of at least 500 and up to 200,000, preferably, a molecular weight distribution of 2.5 or lower, a head-to-tail monomer orientation of 98 percent or higher, total branching of 1 percent or lower, total disordered stereotacticity of 70 percent or lower and a preferred degree of hydrolysis of 98 percent or greater.

In another embodiment, there is a physiologically and mechanically biocompatible artificial pancreas. The pancreas comprises a combination of the hydrogel as set forth just Supra combined with cells. The cells are selected from the group consisting of islets; clusters of islets; Beta cells, and combinations of islets and Beta cells.

At least one more embodiment of this invention is a physiologically and mechanically biocompatible artificial pancreas as set just Supra wherein the pancreas contains vascular grafts thereon or therein.

In a further embodiment, there is a physically crosslinked polyvinyl alcohol hydrogel wherein the hydrogel is formed into a configuration selected from the group consisting of sheets, ribbons, tapes, fibers, threads, rods, tubes, microcapsules, coatings, and combinations of these configurations.

Yet in another embodiment there is a physically crosslinked polyvinyl alcohol hydrogel wherein the hydrogel is formed into a pouch manufactured from a hydrogel selected from the group consisting of sheets, ribbons, tapes, fibers, threads, rods, and tubes.

Still another embodiment of this invention is a physically crosslinked polyvinyl alcohol water-free solid prepared using mixed solvents, the solid being essentially free of water and having a syndiotacticity of fifty percent or greater.

An additional embodiment of this invention is a physiologically and mechanically biocompatible artificial pancreas, therein the pancreas comprises a combination of the water-free solid as set forth just Supra, and cells. The cells are selected from the group consisting of islets; clusters of islets; Beta cells, and combinations of islets and Beta cells.

One more embodiment of this invention is a physiologically and mechanically biocompatible artificial pancreas as set just Supra wherein the pancreas contains vascular grafts thereon or therein.

There are several embodiments regarding the methods and manner in which the BAPs of this invention are manufactured.

For example, there is a method of providing a physiologically and mechanically biocompatible artificial pancreas wherein the method comprises providing a physically crosslinked polyvinyl alcohol hydrogel prepared using mixed solvents, containing continuous, uninterrupted immobilized water wherein the hydrogel has at least 88% by weight water content or higher and has a syndiotacticity of fifty percent or greater. Treating the hydrogel with cells selected from the group consisting of islets; clusters of islets; Beta cells, and combinations of islets and Beta cells. Then, attaching at least one suture to the physiologically and mechanically biocompatible artificial pancreas. By using the word “then” it is not meant that there is any special order with regard to when the sutures are attached.

A second method of providing a physiologically and mechanically biocompatible artificial pancreas wherein the method comprises providing a physically crosslinked polyvinyl alcohol hydrogel prepared using mixed solvents, containing continuous, uninterrupted immobilized water wherein the hydrogel has at least 88% by weight water content or higher and a syndiotacticity of fifty percent or greater. Then treating the hydrogel with cells selected from the group consisting of islets; clusters of islets; Beta cells, and combinations of islets and Beta cells. Thereafter, forming a pouch of the physically crosslinked polyvinyl alcohol hydrogel and sealing the edges of the pouch, and then, attaching at least one suture to the physiologically and mechanically biocompatible artificial pancreas.

Yet another of method of providing a physiologically and mechanically biocompatible artificial pancreas comprises providing a physically crosslinked polyvinyl alcohol water-free solid prepared using mixed solvents, said solid being essentially free of water and having a syndiotacticity of fifty percent or greater. Then, treating the hydrogel with cells selected from the group consisting of islets; clusters of islets; Beta cells, and combinations of islets and Beta cells and then attaching at least one suture to the physiologically and mechanically biocompatible artificial pancreas.

In a further method of providing a physiologically and mechanically biocompatible artificial pancreas, the method comprises providing a physically crosslinked polyvinyl alcohol water-free solid prepared using mixed solvents, said solid being essentially free of water and having a syndiotacticity of fifty percent or greater. Then one treats the hydrogel with cells selected from the group consisting of islets; clusters of islets; Beta cells, and combinations of islets and Beta cells and then forming a pouch of the physically crosslinked polyvinyl alcohol hydrogel and thereafter sealing the pouch along the pouch edges, and thereafter, attaching at least one suture to the physiologically and mechanically biocompatible artificial pancreas.

In still another method of providing a physiologically and mechanically biocompatible artificial pancreas, said method comprises providing a physically crosslinked polyvinyl alcohol hydrogel prepared using mixed solvents, containing continuous, uninterrupted immobilized water and having a syndiotacticity of fifty percent or greater. The hydrogel has at least 88% by weight water content or higher. Thereafter, forming a pouch of the physically crosslinked polyvinyl alcohol hydrogel and inserting cells into the interior of the pouch wherein the cells are selected from the group consisting of islets; clusters of islets; Beta cells, and combinations of islets and Beta cells.

Thereafter, one seals the edges of the pouch and attaches at least one suture to the physiologically and mechanically biocompatible artificial pancreas.

Another embodiment of this invention is a method of providing a physiologically and mechanically biocompatible artificial pancreas wherein the method comprises providing a physically crosslinked polyvinyl alcohol water-free solid prepared using mixed solvents, wherein the solid is essentially free of water and having a syndiotacticity of fifty percent or greater. Then, forming a pouch of the physically crosslinked polyvinyl alcohol hydrogel and thereafter treating the interior of the pouch with cells selected from the group consisting of islets; clusters of islets; Beta cells, and combinations of islets and Beta cells. The pouch is then sealed along the pouch edges and at least one suture is attached to the physiologically and mechanically biocompatible artificial pancreas.

A further embodiment of this invention is a method of pre-vascularization and control of inflammatory activity using a surgically created insertion pocket for a biocompatible artificial pancreas in a living tissue. The method comprises surgically creating a pocket in living tissue. Preloading a hydrogel sheet with a growth factor material and anti-inflammatory bioactive molecules, wherein the hydrogel sheet is a physically crosslinked polyvinyl alcohol hydrogel prepared using mixed solvents, containing continuous, uninterrupted immobilized water wherein the hydrogels have at least 88% by weight water content or higher and a syndiotacticity of greater than fifty percent. Thereafter, inserting the hydrogel sheet into the pocket. Then, providing a surgical closure to the pocket. One then causes vascularization through angiogenesis and healing of the live tissue using a controlled and sequential release of substances that promote vascularization of the pocket. One then causes healing of the live tissue using a controlled and sequential release of wound healing substances into the pocket.

There is a method of placing a biocompatible artificial pancreas in a living tissue. The method comprises surgically creating a pocket in living tissue then preloading a hydrogel sheet with a growth factor material and anti-inflammatory bioactive molecules, wherein the hydrogel sheet is a physically crosslinked polyvinyl alcohol hydrogel prepared using mixed solvents, containing continuous, uninterrupted immobilized water, wherein the hydrogels have at least 88% by weight water content or higher and a syndiotacticity of fifty percent or greater. Thereafter the hydrogel is inserted into the pocket and a surgical closure to made in the pocket the pocket is caused to heal the live tissue using a controlled and sequential release of wound healing substances into the pocket. Thereafter, vascularization is created through angiogenesis of the live tissue using a controlled and sequential release of substances that promote vascularization of said pocket.

Thereafter a protocol is provided to activate angiogenesis to form a vascular plexus in the tissue of the pocket. After a predetermined amount of time the hydrogel sheet is surgically removed.

At this point, a biocompatible artificial pancreas is inserted into the pocket and a surgical closure is provided to the pocket. The biocompatible artificial pancreas comprises a physiologically and mechanically biocompatible artificial pancreas, wherein the biocompatible artificial pancreas comprises an immunoisolating enclosure manufactured from the hydrogel combined with cells, wherein the cells are selected from the group consisting of islets; clusters of islets; Beta cells, and combinations of islets and Beta cells.

Another embodiment of this invention is a method of providing a physiologically and mechanically biocompatible artificial pancreas. The method comprises providing a physically crosslinked polyvinyl alcohol hydrogel prepared using mixed solvents, containing continuous, uninterrupted immobilized water, wherein the hydrogel has at least 88% by weight water content or higher and a syndiotacticity of fifty percent or greater.

Thereafter, treating the hydrogel with cells selected from the group consisting of islets; clusters of islets; Beta cells, and combinations of islets and Beta cells and inserting the physically crosslinked polyvinyl alcohol hydrogel into a blood vessel. One the anastomoses the blood vessel to a part of the vascularization of the hydrogel. One then attaches at least one suture to the physiologically and mechanically biocompatible artificial pancreas. It is contemplated within the scope of this invention to attach sutures to the hydrogel prior to insertion into the blood vessel.

Still, an additional embodiment of this invention is A method of providing a physiologically and mechanically biocompatible artificial pancreas. The method comprises providing a physically crosslinked polyvinyl alcohol water-free solid prepared using mixed solvents, said solid being essentially free of water and having a syndiotacticity of fifty percent or greater and then treating the hydrogel with cells selected from the group consisting of islets; clusters of islets; Beta cells, and combinations of islets and Beta cells and then inserting the treated hydrogel into a blood vessel.

Thereafter, anastomosing said blood vessel to a part of any vascularization of the hydrogel and attaching at least one suture to the physiologically and mechanically biocompatible artificial pancreas.

It is contemplated within the scope of this invention to attach sutures to the hydrogel prior to insertion into the blood vessel.

Also contemplated within the scope of this invention are the use of 3-D tissue printing of the BAP, the placement of the BAP directly into a blood vessel, the placement of catenated, that is, chained, micro- and macro-encapsulation beads, and methods to increase the surface to volume ratio such as the use of corrugated sheets or comb-like structures forming the BAP.

“Low temperature for purposes of this invention means from −25° C. to 60° C.

“High temperature” for purposes of this invention means from 70° C. to 180° C.

### BRIEF DESCRIPTION OF THE SEVERAL VIEWS OF THE DRAWING(S)

FIG. 1A is a full top view of a physiologically and mechanically biocompatible artificial pancreas in a pouch configuration made with bulk polyvinyl alcohol hydrogels showing in-place sutures at the corners.

FIG. 1B is a full open-end view of the device of FIG. 1A showing the cells.

FIG. 2 is a full top view of a physiologically and mechanically biocompatible artificial pancreas of this invention manufactured in a reticulated foam configuration.

FIG. 3 is a full top view of a physiologically and mechanically biocompatible artificial pancreas manufactured from a fiber reinforced flat sheet.

FIG. 4A is a full top view of a flat sheet of a physiologically and mechanically biocompatible artificial pancreas.

FIG. 4B is a full end view of the device of FIG. 4A.

FIG. 5A is a full top view of a built up physiologically and mechanically biocompatible artificial pancreas having structural components to form scaffolding.

FIG. 5B is a full end view of the device of FIG. 5A.

FIG. 6A is a full top view of a physiologically and mechanically biocompatible artificial pancreas showing the use of polyvinyl alcohol of this invention as vascular grafts in non-reinforced BAP.

FIG. 6B is a full top view of a physiologically and mechanically biocompatible artificial pancreas showing the use of polyvinyl alcohol of this invention as vascular grafts in reinforced BAP.

FIG. 7 shows a full top view of a physiologically and mechanically biocompatible artificial pancreas with multiple vascular grafts on the surface of the BAP that is non-reinforced.

FIG. 8A shows a full top view of a physiologically and mechanically biocompatible artificial pancreas containing multiple artificial vascular grafts internally wherein the BAP is not reinforced.

FIG. 8B shows a full top view of a physiologically and mechanically biocompatible artificial pancreas containing multiple artificial vascular grafts internally wherein the BAP is reinforced.

FIG. 9A is a full top view of a physiologically and mechanically biocompatible artificial pancreas showing a single blood vessel that is brought to the BAP. This BAP is non-reinforced.

FIG. 9B is a full top view of a physiologically and mechanically biocompatible artificial pancreas showing a single blood vessel that is brought to the BAP. This BAP is reinforced.

FIG. 10A shown a physiologically and mechanically biocompatible artificial pancreas in a larger oval shape that is non-reinforced.

FIG. 10B is a physiologically and mechanically biocompatible artificial pancreas in a larger oval shape that is fiber or cloth reinforced.

FIG. 11A is a full top view of a smaller physiologically and mechanically biocompatible artificial pancreas that is not reinforced.

FIG. 11B is a full to view of a smaller physiologically and mechanically biocompatible artificial pancreas that is reinforced.

FIG. 12A is a full top view of a smaller physiologically and mechanically biocompatible artificial pancreas showing vascular grafts thereon wherein the BAP is non-reinforced.

FIG. 12B is a full top view of a smaller physiologically and mechanically biocompatible artificial pancreas showing vascular grafts thereon wherein the BAP is reinforced.

FIG. 13A is a full top view of a circular physiologically and mechanically biocompatible artificial pancreas that is not reinforced.

FIG. 13B is a full top view of a circular physiologically and mechanically biocompatible artificial pancreas that is reinforced.

FIG. 14A is a full side view of a round tubular physiologically and mechanically biocompatible artificial pancreas with a center opening showing a vascular graft wherein the BAP is not reinforced.

FIG. 14B is a full end view of a round tubular physiologically and mechanically biocompatible artificial pancreas with a center opening showing a vascular graft wherein the BAP is not reinforced.

FIG. 15A is a full side view of a round tubular physiologically and mechanically biocompatible artificial pancreas with a center opening showing a vascular graft wherein the BAP is reinforced.

FIG. 15B is a full end view of a round tubular physiologically and mechanically biocompatible artificial pancreas with a center opening showing a vascular graft wherein the BAP is reinforced.

FIG. 16A is full side view of an oval tubular physiologically and mechanically biocompatible artificial pancreas with a center opening showing a vascular graft wherein the BAP is not reinforced.

FIG. 16B is a full end view of an oval tubular physiologically and mechanically biocompatible artificial pancreas with a center opening showing a vascular graft wherein the BAP is not reinforced.

FIG. 17A is a full side view of an oval tubular physiologically and mechanically biocompatible artificial pancreas with a center opening showing a vascular graft wherein the BAP is reinforced.

FIG. 17B is a full end view of an oval tubular physiologically and mechanically biocompatible artificial pancreas with a center opening showing a vascular graft wherein the BAP is reinforced.

FIG. 18A shows a full top view of a ribbon configuration of a physiologically and mechanically biocompatible artificial pancreas wherein the BAP is not reinforced.

FIG. 18B shows a full top view of a ribbon configuration of a physiologically and mechanically biocompatible artificial pancreas wherein the BAP is reinforced.

FIG. 18C shows a full top view of a ribbon configuration of a physiologically and mechanically biocompatible artificial pancreas wherein the BAP is a hydrogel foam.

FIG. 19A is full side view of a solid tubular physiologically and mechanically biocompatible artificial pancreas wherein the BAP is not reinforced.

FIG. 19B is a full end view of a solid tubular physiologically and mechanically biocompatible artificial pancreas wherein the BAP is not reinforced.

FIG. 20A is a full side view of a solid tubular physiologically and mechanically biocompatible artificial pancreas wherein the BAP is reinforced.

FIG. 20B is a full end view of a solid tubular physiologically and mechanically biocompatible artificial pancreas wherein the BAP is reinforced.

FIG. 21A is a full side view of a solid oval tubular physiologically and mechanically biocompatible artificial pancreas wherein the BAP is not reinforced.

FIG. 21B is a full end view of a solid oval tubular physiologically and mechanically biocompatible artificial pancreas wherein the BAP is not reinforced.

FIG. 22A is a full side view of a solid tubular physiologically and mechanically biocompatible artificial pancreas wherein the BAP is reinforced.

FIG. 22B is a full end view of a solid tubular physiologically and mechanically biocompatible artificial pancreas wherein the BAP is reinforced.

FIG. 23A is a full side view of a solid rod-like physiologically and mechanically biocompatible artificial pancreas wherein the BAP is not reinforced.

FIG. 23B is a full end view of a solid rod-like physiologically and mechanically biocompatible artificial pancreas wherein the BAP is not reinforced.

FIG. 24A is a full side view of a solid oval rod-like physiologically and mechanically biocompatible artificial pancreas wherein the BAP is not reinforced.

FIG. 24B is a full end view of a solid oval rod-like physiologically and mechanically biocompatible artificial pancreas wherein the BAP is not reinforced.

FIG. 25 is a full side view of a circular or rod-like physiologically and mechanically biocompatible artificial pancreas wherein the BAP is manufactured from hydrogel foam.

FIG. 26A is a solid fiber or thread BAP.

FIG. 26B is a hollow fiber or thread BAP.

FIG. 27 is a full view of a chain of spherical encapsulated BAP.

FIG. 28 is a pancreas of this invention in a convoluted shape for more surface area.

FIG. 29 is a comb-like configuration of a pancreas of this invention.

### DETAILED DESCRIPTION OF THE INVENTION

This invention deals in one embodiment with novel polyvinyl alcohol hydrogels and solids and their use in providing physiologically and mechanically biocompatible artificial pancreas.

“Biocompatibility” used in this invention is used only as a general term because biocompatibility is not precisely defined in the field. Biocompatibility means different things for different applications such as cardiovascular, cosmetic surgery, ophthalmic work, and so on.

Thus, herein, the inventor addresses distinct families of biocompatibility requirements for biomaterials, that is, “physiological” and “mechanical” biocompatibility. A better understanding of biocompatibility and a need for more precise requirements relates to biocompatibility that is driven by the development of non-immunogenic biomaterials of the present invention.

The biomaterials of the instant invention have a non-immunogenic response, that is, the family of biomaterials on the instant invention deprives the human immune system of having cellular and molecular mechanisms to identify surfaces of biomaterial of the present invention as foreign bodies.

The overall objective of the instant invention is the development of a long-term solution to restore the body of a human to produce not only insulin but also other accompanying hormones and bioactive molecules that are critical for health, and prevention of secondary health issues as they may become serious problems when a diabetic person uses insulin injections for a long term.

Hence, the ultimate objective of the instant invention is the development of a bioartificial pancreas (BAP) that possess an immune isolation membrane that can allow the implantation of a BAP into a blood vessel, which provides all the known functions of islets; implantation of BAP into other tissues that can provide sufficient blood supply to the outer surface of the BAP using well-developed vascular plexus and use BAP to house islets or beta cells, in immune-isolation blood that will be circulated using vascular grafts made from the biomaterials of the present invention.

There are different modes of operation and different uses of the family of PVA-based hydrogels made using mixed solvents whose properties are fully customizable allowing practically an infinite number of PVA hydrogels. Biomaterials have specific single or multiple gradient properties.

Surface properties of all kinds of PVA-based biomaterials can be customized using a wide range of parameters to arrive at surface properties that closely resemble native properties of human tissue as well as providing scaffolding that will support and drive tissue engineering as part of the medicine carried out in the desired direction of the present invention. This is accomplished by a combination of activities provided by such full customizable scaffolding such as customizing physical/mechanical properties so that cells can adhere and receive the desired signaling based on hardness of the surface and strength of adhesion.

Surface modification to provide desired levels of physiological biocompatibility so that surfaces of non-immunological biomaterial assume certain desired levels of visibility to the cells and to the immune system so that adhesion and cell migration can take place.

Surface modification can take place in any desired way to direct migration of cells in a preferred direction by custom modification of the hardness and adhesion to provide the migration path of the cells. Use controlled release capability of the hydrogel that can benefit from the gradient properties for the hydrogel which can direct the rate and direction of the release of growth factors, chemokines, cytokines, and other physiologically and thus cellularly active entities to precisely guide reversative processes as well as re-establishing homeostasis of transplanted cells or transplanted entire tissue such as cells in addition-to the Extracellular Matrix (ECM) which is important in transplantation of individual and clusters of islets as well as imbedded beta cells.

Thus, the BAP's of this invention can be made in various thicknesses, water content, levels of single or gradient levels of biostability with different rates of bioerosion, levels of biocompatibility that includes physiological and mechanical biocompatibility, tensile strength, a degree of reinforcement that is typically done using long fibers in the form of mats of woven and non-woven fibrous structures, the use of biostable fibers like PVA and polyethylene (PE), with modified surfaces, to form hydrogen bonds, the use of biodegradable fibers such as those made from biodegradable polymers including collagen and other similar options. PVA fibers as well as silk and fibers based on surface modified polymers such as PE are used for reinforcement and sutures. Cyanoacrylate glues can be used to seal and to immobilize the BAP, for example, when pouches and the like are used.

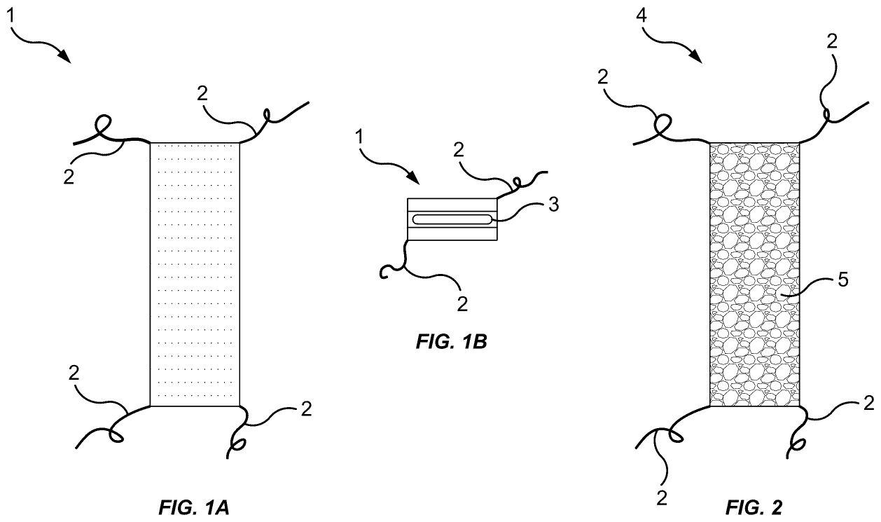

Turning now to FIG. 1A, there is shown a full top view of a physiologically and mechanically biocompatible artificial pancreas 1 in a pouch configuration made with bulk polyvinyl alcohol hydrogels showing in-place sutures 2 at the corners. FIG. 1B is a full open-end view of the device of FIG. 1A showing the cells 3.

The hydrogels used to make the pouch are two thin sheets having a thickness of about 50 mm or thicker. All sheets at this thickness have good mechanical strength and provide a safe operation of the device with a high burst strength.

A first hydrogel sheet is placed on a solid surface and then the cells are formed on top of the sheet. Then, the second sheet is placed on top of the cells. The sheets are then sealed at the edges to form the pouch. The sutures are thereafter attached, and the open end of the pouch is sealed along the edges. The end sealing and attachment of the sutures can be done by using cyanoacrylate glue, or by using warm to hot dilute solutions of the PVA (from 3 to 8 weight percent PVA in mixed solvent) from which the sheets have been formed, or by implantation of the sutures.

The overall dimensions of the device are not critical as long as one takes note of the type of device, its intended location when implanted, and the manner of implantation. Typically, the devices of this nature are about 120 mm in length, 80 mm in width and the thickness is about 0.150 mm on average.

The manner in which the sealing is done is not critical to the invention as long as the pouches are securely sealed, and any puncture holes are also sealed.

This same method, generally, is used in forming all of the materials within the scope of this invention.

Solid PVA materials and low water content PVA hydrogels can gain their non-immuno-genic characteristics after their surface is modified to contain water above 88%. Modification of low water content PVA surfaces to a high-water content hydrogel surface can be accomplished by coating the surfaces with essentially monomolecular layers of high-water content hydrogels, that is, hydrogels having water contents above 88%. The instant invention converts the surface of any material to behave as if it is a true high-water content PVA hydrogel and thus, behaves as if it is non-immunogenic. This process is also important for the conversion of existing medical devices to devices that exhibit non-immunogenic behavior.

The order of steps in the methods and processes disclosed and claimed herein are not critical with the exception of, order of steps created by the steps themselves, which are reasonable.

Various geometric shapes and configurations of the devices can be made by this invention. For example, pouches, see FIGS. 1A and 1B showing the artificial pancreas 1 with sutures 2 and cells 3. FIG. 1B is a full end view of FIG. 1A showing the pancreatic cell location.

FIG. 2 is a full top view of a physiologically and mechanically biocompatible artificial pancreas 4 of this invention manufactured in a reticular configuration, nominally foam 5.

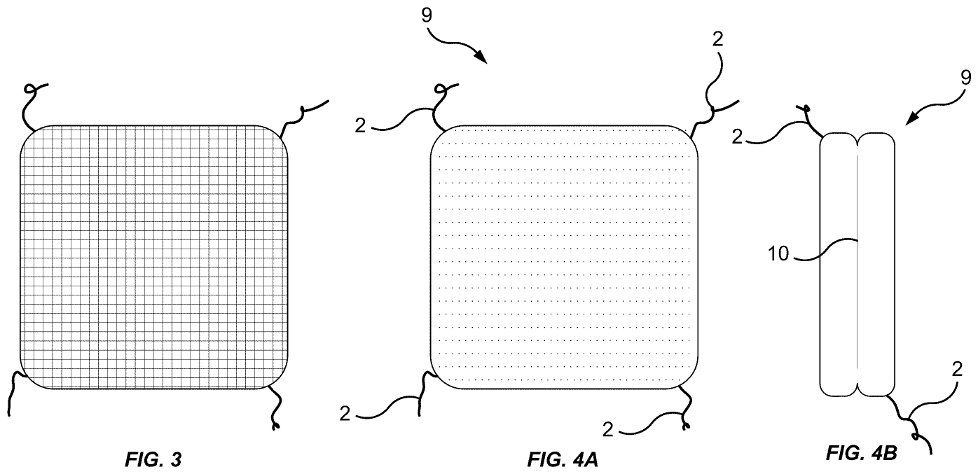

FIG. 3 [symbol] is a full top view of a physiologically and mechanically biocompatible artificial pancreas 6 manufactured from a flat sheet. The device 6 is shown with half of the surface covered with long fibers 7 and half with woven fibers 8 used as reinforcement. This is not the normal mode, but in this case is used for illustration purposes.

FIG. 4A is a full top view of a flat sheet of a physiologically and mechanically biocompatible artificial pancreas 9 shown without reinforcement. FIG. 4B is a full end view of FIG. 4A showing the sealed end 10.

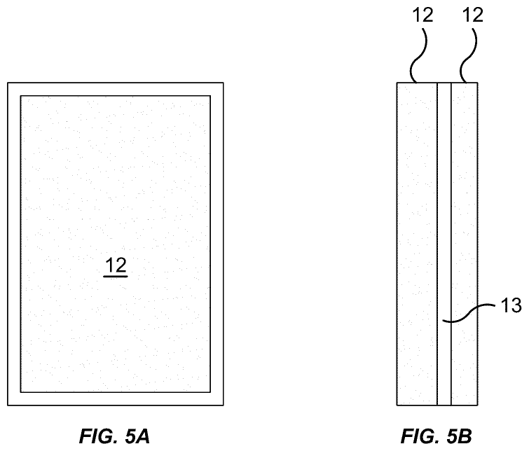

FIG. 5A is a full top view of a built up physiologically and mechanically biocompatible artificial pancreas 11 showing a hydrogel membrane 13 and having structural components to form scaffolding 12. FIG. 5B is a full end view of the device of FIG. 5A.

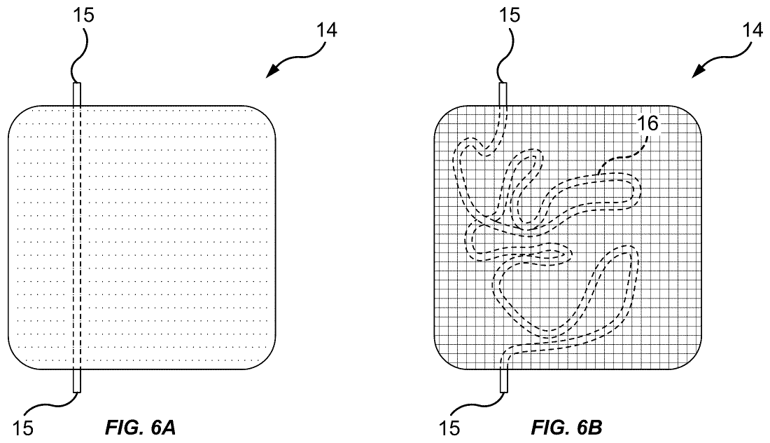

FIG. 6A is a full top view of a physiologically and mechanically biocompatible artificial pancreas 14 showing the use of polyvinyl alcohol of this invention as vascular grafts 15 in non-reinforced BAP.

FIG. 6B is a full top view of a physiologically and mechanically biocompatible artificial pancreas showing the use of polyvinyl alcohol of this invention as vascular grafts 16 in reinforced BAP.

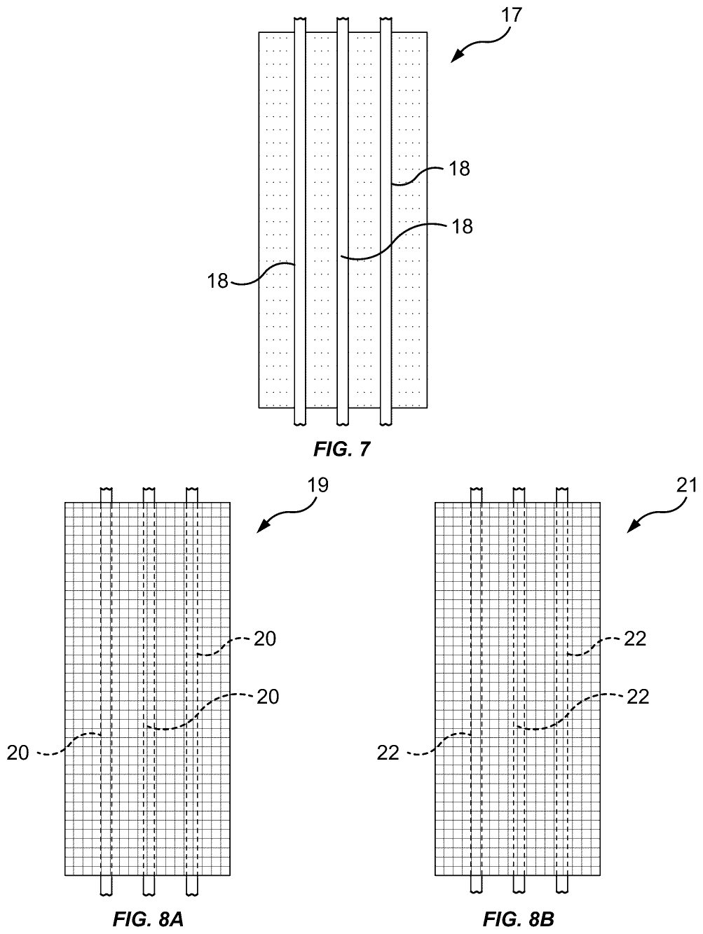

FIG. 7 shows a full top view of a physiologically and mechanically biocompatible artificial pancreas 17 with multiple vascular grafts 18 on the surface of the BAP that is non-reinforced.

FIG. 8A shows a full top view of a physiologically and mechanically biocompatible artificial pancreas 19 containing multiple artificial vascular grafts 20 internally wherein the BAP is not reinforced.

FIG. 8B shows a full top view of a physiologically and mechanically biocompatible artificial pancreas 21 containing multiple artificial vascular grafts 22 wherein the BAP is reinforced.

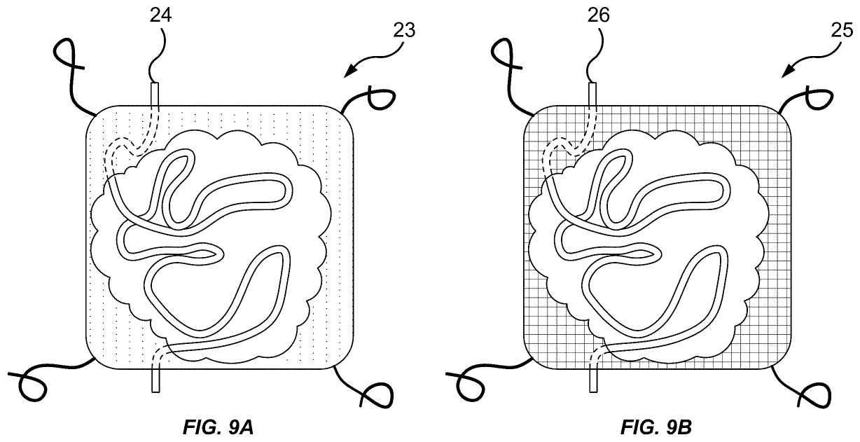

FIG. 9A is a full top view of a physiologically and mechanically biocompatible artificial pancreas 23 showing a single blood vessel 24 that is brought to the BAP. This BAP is non-reinforced.

FIG. 9B is a full top view of a physiologically and mechanically biocompatible artificial pancreas 25 showing a single blood vessel 26 that is brought to the BAP. This BAP is reinforced.

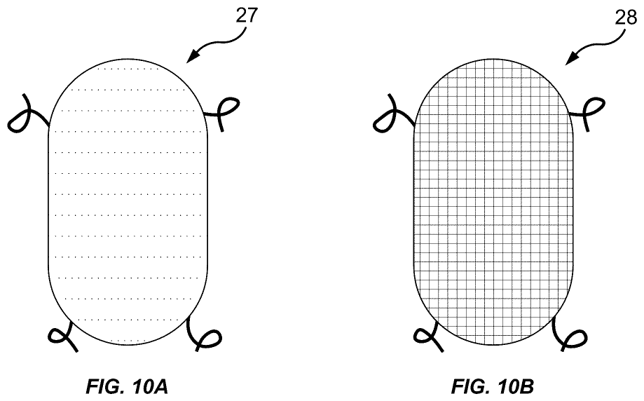

FIG. 10A shown a physiologically and mechanically biocompatible artificial pancreas 27 in a larger oval shape that is non-reinforced. This oval nominally is about 65 mm wide, about 100 mm long, and about 0.150 mm thick.

FIG. 10B is a physiologically and mechanically biocompatible artificial pancreas 28 in a larger oval shape that is reinforced. This oval nominally is about 75 mm wide, about 115 mm long and about 0.050 mm thick.

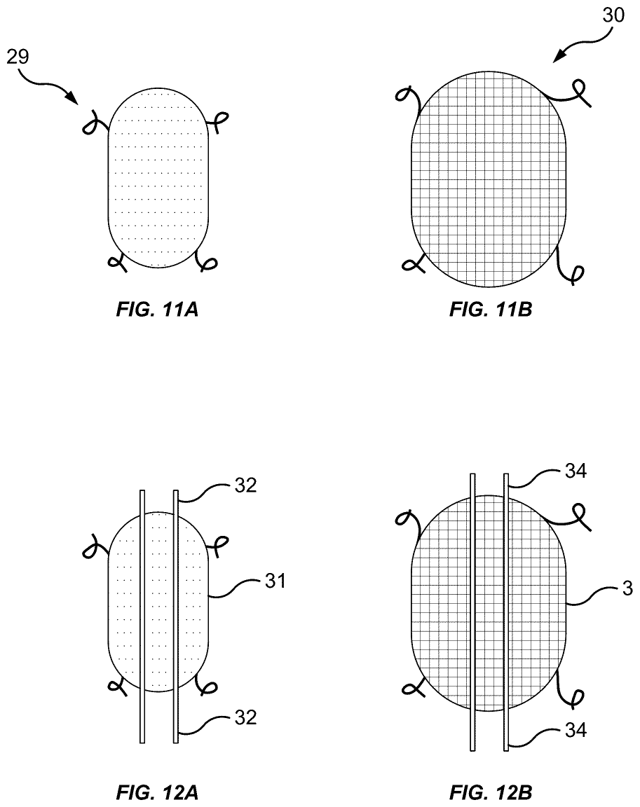

FIG. 11A is a full top view of a smaller physiologically and mechanically biocompatible artificial pancreas 29 that is not reinforced. This oval is about 25 mm wide, about 50 mm long and about 0.150 mm thick.

FIG. 11B is a full top view of a smaller physiologically and mechanically biocompatible artificial pancreas 30 that is reinforced. This oval is about 30 mm wide, about 55 mm long, and about 0.050 mm thick.

FIG. 12A is a full top view of a smaller physiologically and mechanically biocompatible artificial pancreas 31 showing vascular grafts 32 placed thereon wherein the BAP is non-reinforced.

FIG. 12B is a full top view of a smaller physiologically and mechanically biocompatible artificial pancreas 33 showing vascular grafts 34 placed thereon wherein the BAP is reinforced.

FIG. 13A is a full top view of a circular physiologically and mechanically biocompatible artificial pancreas 35 that is not reinforced. This sheet has a radius of about 100 mm and a thickness of about 0.150 mm.

FIG. 13B is a full top view of a circular physiologically and mechanically biocompatible artificial pancreas 36 that is reinforced. This sheet has about a 30% larger surface area than 13B to accommodate for the loss of diffusion area due to the presence of reinforcement.

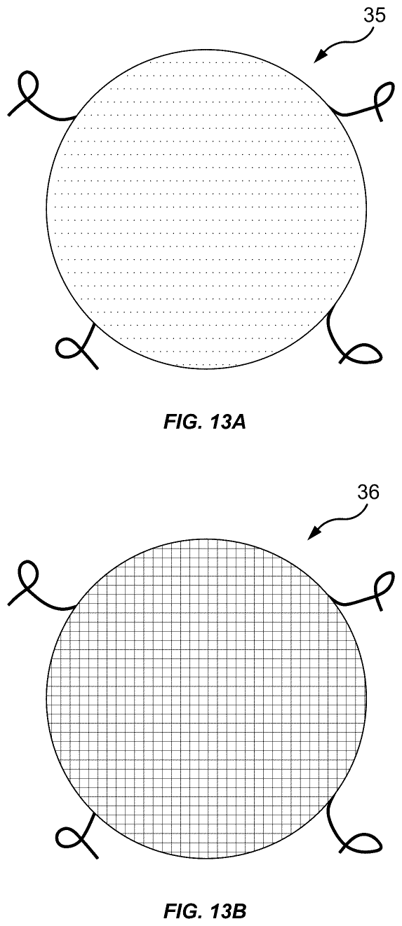

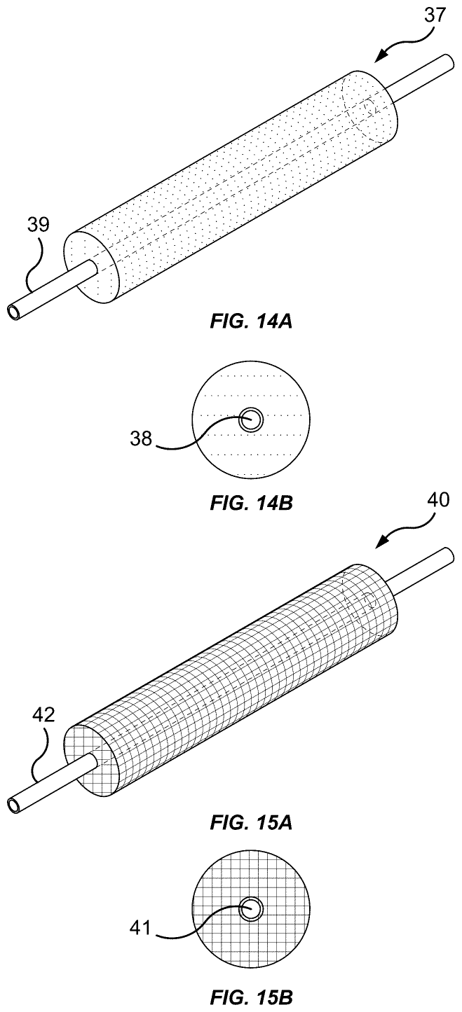

FIG. 14A is a full side view of a round tubular physiologically and mechanically biocompatible artificial pancreas 37 with a center opening 38 showing a vascular graft 39 wherein the BAP is not reinforced. The radius of this configuration is about 6 mm, and the length of the tube is about 100 mm wherein the vascular graft has an inside diameter of about 2 mm and the wall thickness is about 0.5 mm.

FIG. 14B is a full end view of a round tubular physiologically and mechanically biocompatible artificial pancreas of FIG. 14A with a center opening 38 showing a vascular graft 39 wherein the BAP is not reinforced.

FIG. 15A is a full side view of a round tubular physiologically and mechanically biocompatible artificial pancreas 40 with a center opening 41 showing a vascular graft 42 wherein the BAP is reinforced.

FIG. 15B is a full end view of a round tubular physiologically and mechanically biocompatible artificial pancreas of FIG. 15A with a center opening 41 showing a vascular graft 42 wherein the BAP is reinforced.

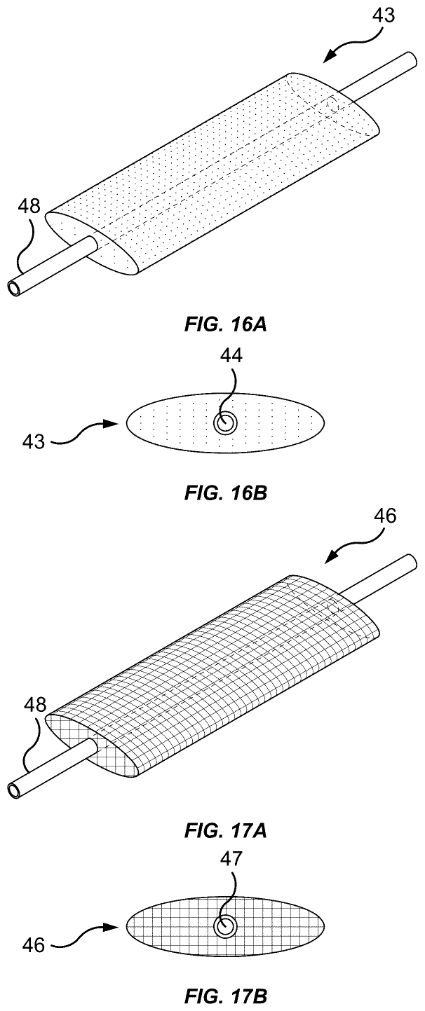

FIG. 16A is full side view of an oval tubular physiologically and mechanically biocompatible artificial pancreas 43 with a center opening 44 and a vascular graft 45 wherein the BAP is not reinforced.

FIG. 16B is a full end view of an oval tubular physiologically and mechanically biocompatible artificial pancreas 43 with a center opening 44 showing a vascular graft 45 wherein the BAP is not reinforced.

FIG. 17A is a full side view of an oval tubular physiologically and mechanically biocompatible artificial pancreas 46 with a center opening 47 showing a vascular graft 48 wherein the BAP is reinforced.

FIG. 17B is a full end view of an oval tubular physiologically and mechanically biocompatible artificial pancreas 46 with a center opening 47 showing a vascular graft 48 wherein the BAP is reinforced.

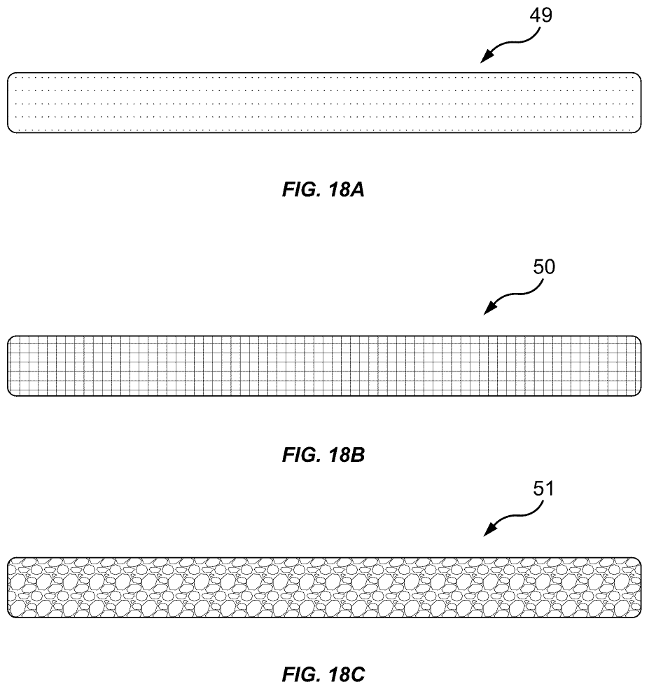

FIG. 18A shows a full top view of a ribbon configuration of a physiologically and mechanically biocompatible artificial pancreas 49 wherein the BAP is not reinforced.

FIG. 18B shows a full top view of a ribbon configuration of a physiologically and mechanically biocompatible artificial pancreas 50 wherein the BAP is reinforced.

FIG. 18C shows a full top view of a ribbon configuration of a physiologically and mechanically biocompatible artificial pancreas 51 wherein the BAP is a reticulated hydrogel foam.

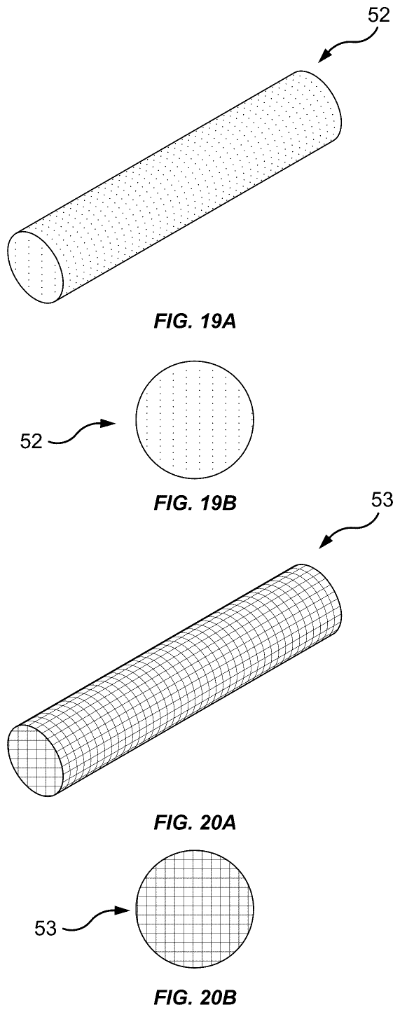

FIG. 19A is full side view of a tubular physiologically and mechanically biocompatible artificial pancreas 52 wherein the BAP is not reinforced which houses islets within the inner tubular space.

FIG. 19B is a full end view of a tubular physiologically and mechanically biocompatible artificial pancreas 52 wherein the BAP is not reinforced, which houses islets within the inner tubular space.

FIG. 20A is a full side view of a tubular physiologically and mechanically biocompatible artificial pancreas 53 wherein the BAP is reinforced, which houses islets within the inner tubular space.

FIG. 20B is a full end view of a tubular physiologically and mechanically biocompatible artificial pancreas 53 wherein the BAP is reinforced, which houses islets within the inner tubular space.

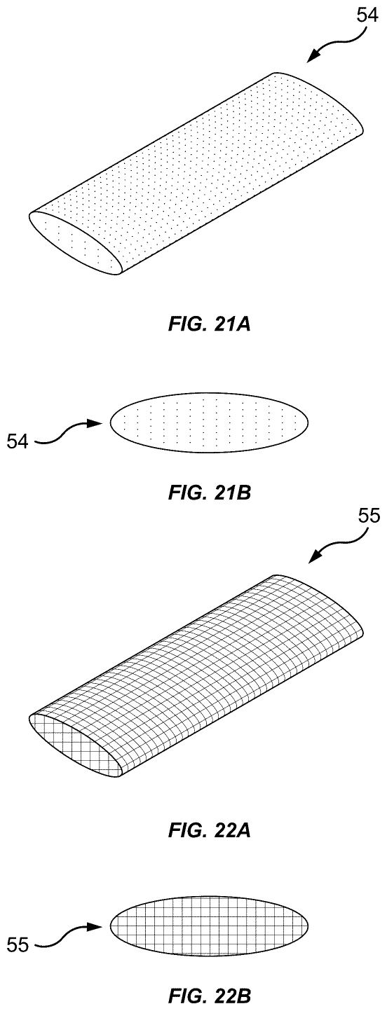

FIG. 21A is a full side view of a solid oval tubular physiologically and mechanically biocompatible artificial pancreas 54 wherein the BAP is not reinforced.

FIG. 21B is a full end view of an oval tubular physiologically and mechanically biocompatible artificial pancreas 54 wherein the BAP is not reinforced.

FIG. 22A is a full side view of an oval tubular physiologically and mechanically biocompatible artificial pancreas 55 wherein the BAP is reinforced.

FIG. 22B is a full end view of a oval physiologically and mechanically biocompatible artificial pancreas 55 wherein the BAP is reinforced.

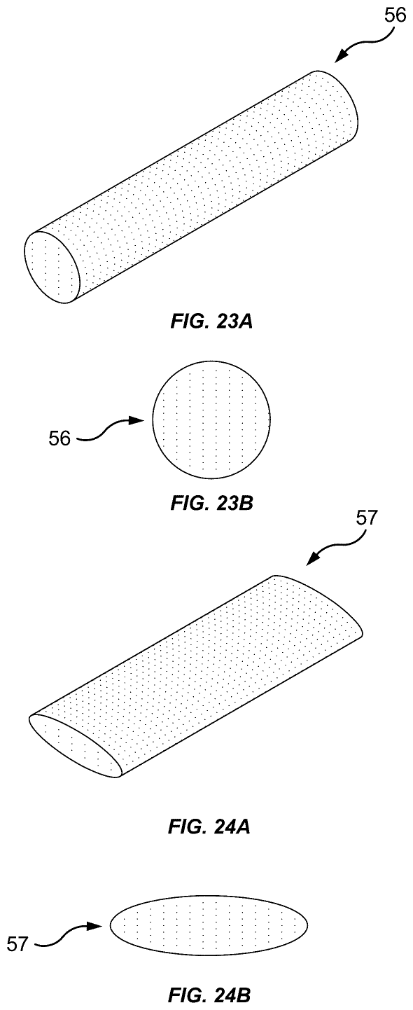

FIG. 23A is a full side view of a solid circular rod-like physiologically and mechanically biocompatible artificial pancreas 56 wherein the BAP is not reinforced.

FIG. 23B is a full end view of a solid rod-like physiologically and mechanically biocompatible artificial pancreas 56 wherein the BAP is not reinforced.

FIG. 24A is a full side view of a solid oval rod-like physiologically and mechanically biocompatible artificial pancreas 57 wherein the BAP is reinforced.

FIG. 24B is a full end view of a solid oval rod-like physiologically and mechanically biocompatible artificial pancreas 57 wherein the BAP is reinforced.

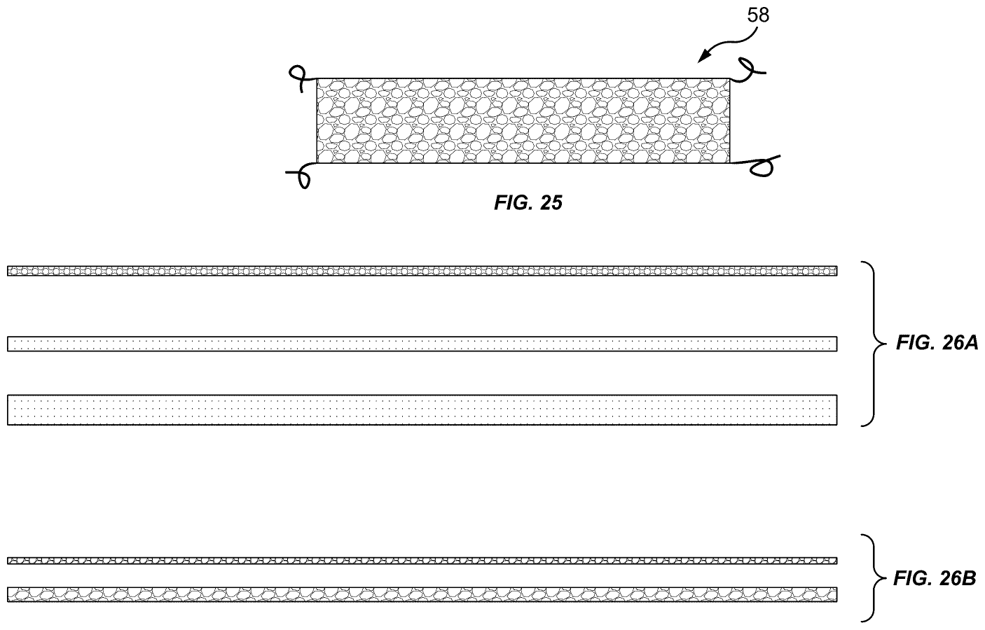

FIG. 25 is a full side view of a circular or rod-like physiologically and mechanically biocompatible artificial pancreas 58 wherein the BAP is manufactured from a reticulated hydrogel foam.

FIG. 26A is a solid fiber or solid thread BAP.

FIG. 26B is a hollow fiber or hollow thread BAP.

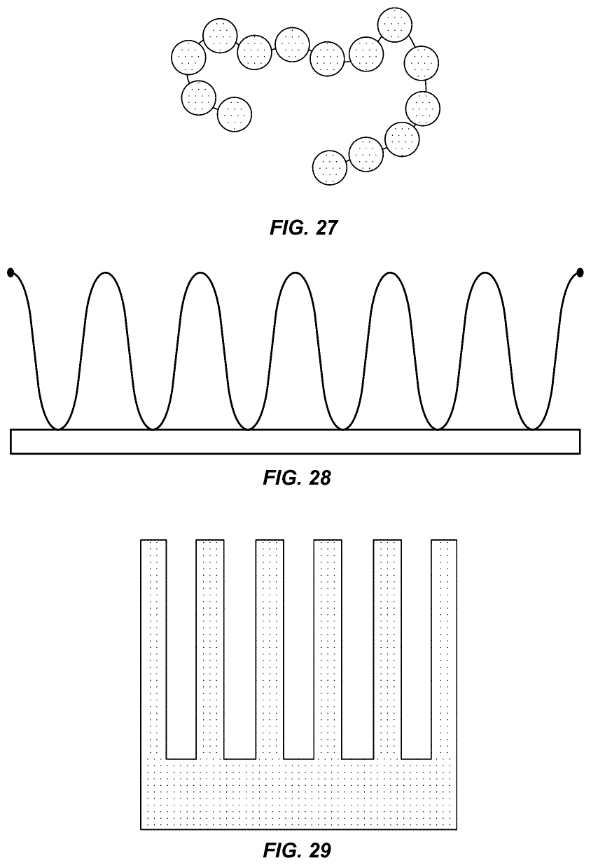

FIG. 27 is a full view of a chain of spherical encapsulated BAP.

FIG. 28 is a full end view of a corrugated hydrogel with cells being place into enclosed channels.

FIG. 29 is a comb-like structure of hydrogel with cells being embedded into a PVA hydrogel.

### EXAMPLES

When used herein, “low viscosity” means 200 cps to 1,000,000 cps at 25°. When used herein, “low temperature” means −35° C. to 40° C.

When used wherein, “pore size” or “reticulation” refers to a pore size ranging from 0.5 to 5 microns. 1 micron equals 10,000 Anstrom units or 1 Angstrom unit equals 0.00001 microns.

### Example 1. Examples of Biomaterial Preparation

This example deals with making enzymatically stable PVA Hydrogels having pore sizes suitable for making immune-isolation pouches that house pancreatic cells in the form known as Bioartificial Pancreas (BAP). Hydrogels are made using mixed solvents based on dimethyl sulfoxide (DMSO) and other solvents.

A poly(vinyl alcohol) polymer having a 150,000 molecular weight, a viscosity of 67 cps and having 99.95% degree of hydrolysis was dissolved in a mixed solvent composed of 70/30 dimethyl sulfoxide (DMSO) and water. A PVA solution having a concentration of 8% wt./wt. of PVA, was made by weighing 16 gms of PVA in powdery form and placed into a dissolution kettle containing 186 gms of the mixed solvent. Dissolution was carried out at 90° C. in a water bath under a nitrogen atmosphere blanket over the PVA solution while continuously stirring for four hours. This resulted in a low viscosity PVA solution, which is used for making PVA hydrogel bulk and cellular hydrogel sheets by molding, extrusion, casting and 3-D Printing. This PVA solution was subjected to cooling in a refrigerator/freezer at a temperature of −18° C. for 8 hours. It is critical to emphasize that only one cooling cycle is required. The cooling process initiates formation of submicron crystallites that act as crosslinkers and reinforcing agents.

When DMSO is used as co-solvent for making physically crosslinked PVA hydrogels, the DMSO needs to be removed in certain medical applications because it may cause lysis of certain cells especially of red blood cells. DMSO is removed from these hydrogels by submersing these hydrogels into a water bath for 3 hours. Then water is replaced four times every 3 hours for complete removal of DMSO. In another example, running water was used for extraction of DMSO by using 0.5 liter of water/minute flow rate into a submersion bath for a duration of 90 minutes.

This process of preparation of physically crosslinked PVA Hydrogel sheets results in PVA hydrogels that have pores size in the range of 150 to 200 Å. A PVA solution of this example was used to make BAP enclosing objects such as PVA hydrogel pouches made from sheets, ribbons, tapes, fibers. threads, rods, microcapsules and coatings. There hydrogels can be used either non-reinforced, that is, “as is” bulk hydrogel or reinforced by continuous fibers. Pancreatic encapsulating pouches were made from hydrogel sheets having thickness from 20 to 500 μm.

### Example 2

This example deals with making enzymatically stable PVA Hydrogels having pore sizes suitable for making immune-isolation pouches. However, these PVA hydrogels are made using mixed solvents based on Glycerin since glycerin does not have a strong lysis action on cell membranes. In making these PVA hydrogels, glycerin does not need to be removed. Other hydrophilic solvents that do not interfere with lysis of cells can also be used. This example follows the same procedure as outlined in the Example 1 except the following:

The mixed solvent for dissolution of PVA powder was made using glycerin instead of DMSO. The mixed solvent in this example has the following composition: 8% glycerol and 92% water. The same dissolution and physical crosslinking procedures were used here as described in the Example 1. This composition of mixed solvent is used in those cases when PVA solutions are used for tissue printing, that is, when one needs to incorporate living cells into the PVA hydrogel matrix where the an PVA matrix serves not only as immune-isolation matrix but also as an extracellular matrix (ECM) for imbedded pancreatic cells.

### Example 3. Procedure for Making Enzymatically Stable BAP Using Bulk and Cellular PVA Hydrogels

This example deals with making enzymatically stable bulk PVA Hydrogels for embedding pancreatic cells using manufacturing processes such as 3-D tissue printing, tissue extrusion, tissue molding and use of any other known manufacturing procedure that can provide immune-isolation to embedded cells within Bioartificial Pancreas (BAP) without creating destruction to imbedded pancreatic cells caused by the shear or other forces generated during the manufacturing. In this example, only PVA hydrogels made from mixed solvents based on Glycerin or other hydrophilic solvents that are cell membrane friendly, were used to prevent cell lysis.

The same procedure as outlined in Example 2 is used here except the following:

Once glycerin/water solution of PVA has been made by dissolution of PVA powder by heating at about 90° C., the temperature of the PVA solution is lowered to about 40° C. (typically temperatures of solution were between 3° and 45° C.). Pancreatic cells are added into the low viscosity PVA solution in glycerin-based mixed solvent to generate 6% population of pancreatic cells within the PVA solution, and gently mixed to uniformly disperse the cells. During addition of the cells and gentle mixing, the container was blanketed with an atmosphere enriched with oxygen.

This PVA solution made using glycerin-based mixed solvent containing dispersed pancreatic cells was used for manufacturing BAP using 3-D tissue printing, tissue extrusion, tissue molding or similar processing that does not cause destruction of imbedded pancreatic cells in the PVA hydrogel matrix. The continuous PVA hydrogel matrix serves as immune-isolation as well as an Extracellular Matrix (ECM) of pancreatic cells.

It is important to emphasize that these tissue manufacturing processes based on PVA solution made by glycerin as a key component in the mixed solvent are used at temperatures 30° C. to 45° C. or as low as about 10° C. to minimize temperature stress to pancreatic cells and also to slow the metabolism of the cells down to reduce loss of pancreatic cell population.

The resulting encapsulating devices, housing pancreatic cells imbedded in the bulk hydrogel, were made in the form of a single sheet, thin rods, tapes, ribbons and threads etc. In all cases these are non-hollow but solid PVA hydrogel objects.

However, small dimeter tubes having 3 mm ID and 0.5 mm wall thickness were also made. In this case the walls of tubes serve as a housing for pancreatic cells serving two functions as mentioned supra: immuno-isolation and providing bi-directional molecular species exchange.

These small diameter tubular BAPs served also as non-thrombogenic vascular grafts providing “ideal” direct contact between imbedded immune-isolated pancreatic cells within the wall of the tube and the blood stream. This design of BAP makes one of the most desirable designs of BAP of the present invention.

### Example 4

This example references making cellular hydrogels, that is, open cell sponges where the cell walls are fully hydrated, that is, the cell walls are made from true hydrogels, which provide housing for immuno-isolation of pancreatic cells. One must keep in mind that if cell walls of the open cell sponge are collapsed by dehydration either by heating or using submersion into a solvent like isopropyl alcohol (IPA), ethyl alcohol, acetone or similar PVA polymer non-solvent, the resulting cellular walls will collapse and as such cannot support physiological functions of imbedded pancreatic cells.

The same procedure as outlined in Example 3 for making tissue printing structures based on cellular (sponge) configurations was used here except the following:

Once glycerin/water solution of PVA has been made by dissolution of PVA powder by heating at about 90° C., the temperature of the PVA solution is lowered to about 40° C. (typically temperatures of solution were between 3° and 45° C.). Pancreatic cells are added into the low viscosity PVA solution in glycerin-based mixed solvent to generate a 6% population of pancreatic cells within the PVA solution, and solution was gently mixed to uniformly disperse the cells. During addition of the cells and gentle mixing, the container is blanketed with an atmosphere enriched with oxygen.

This PVA solution made using glycerin-based mixed solvent containing dispersed pancreatic cells was used for manufacturing BAP using 3-D tissue printing, tissue extrusion, tissue molding or similar processing that does not create destruction of the imbedded pancreatic cells in the PVA hydrogel matrix. The continuous PVA hydrogel matrix serves as immuno-isolation as well as Extracellular Matrix (ECM) of pancreatic cells.

An open cell sponge structure was obtained by addition of dissolvable pore-forming material of any particle shape like starch or sugar and was gently mixed with PVA solution containing dispersed pancreatic cells. Once pore forming particles were matrixed into PVA solution, the mixture was cooled to accelerate physical crosslinking. Once the system was crosslinked the starch or sugar or similar pore-forming particles are removed by placing the sheets into water. It is not necessary to completely remove sugar or starch since their residual amounts can be removed by metabolization.

Open cell sponges were made using blowing air bubbles and gently mixing. The resulting structure is a porous structure. No surfactant was used to eliminate the potential for cell lysis.

Besides single sheets made of cellular PVA hydrogel, other forms were made using molding and extrusion. One has to ensure that open cell structures are formed as much as possible, the higher the percentage of open cells, the better, because the thinner the open cell walls of the sponge are better. This is because thinner cell walls improve diffusion and thus that aids in an increase of rate of exchange of molecular species and oxygen with immune-isolated pancreatic cells.

### Example 5. Different Configurations of BAP

This example deals with methods of making BAP pouches using two sheets stacked on top of each other to make a flat enclosed structure in the form of rectangles, ovals, circles, ribbons or tapes. The two sheets are cohesively bonded along the edges providing inner spaces in the pouches that is completely sealed at the edges and completely excluding any access from the outer space into the inner space. This creates inner spacing that creates complete an immuno-isolation space for pancreatic cells.

The same procedure as outlined in Example 1 is used here except the following:

The procedure for making PVA solution was outlined in the Example 1. Flat sheets of PVA hydrogel were made by 3-D printing, casting, extrusion, and molding. One can use the sheets either just after being taken from the refrigeration chamber, after crosslinking, or after the sheets were subjected to DMSO extraction. After two sheets are placed on top of each other, one can do one of the following to seal the edges of the BAP pouches in the form required. One can submerse just the edges into warm to hot water (55° C. to 70° C.) for 5 to 10 seconds to dissolve the edges and fuse the edges by creating cohesive bonding between two sheets of PVA hydrogel, or follow the same procedure but using warm to hot dilute hydrogel solutions at about 3 to 5 weight percent of PVA in mixed solvent or in water, or using steam coming from a nozzle to soften and wet or dissolve the edges of the sheets to form a new bulk hydrogel that cohesively seals the edge and forms a pocket or pouch, keeping in mind that in these cases the actual process of sealing is based on dissolution of the edges of hydrogel sheets because one is dealing with a physically crosslinked hydrogel, that is, a PVA network continuing either water or mixed solvent. Thus, one cannot use melt sealing but sealing of dissolved material is acceptable.

### Example 6. Method of Loading Pancreatic Cells, for Example, Islets into the Immunization Space of a Pouch

This example deals with a method of loading pancreatic cells such as islets into the immunization space of a BAP pouch of any form of preparation that is described in Example 5. The loading of pancreatic cells and sealing of the pancreatic cell loading port is described infra.

The same procedure as outlined in Example 5 is used here except for the following. A BAP pouch of any form is made from PVA hydrogel bulk sheets by stacking the sheets on top of each other and sealing the edges. Then, the pancreatic cells, cluster of pancreatic cells, or entire islet community cells, are loaded into to space within the BAP pouch that will serve as an immuno-isolation space and provide unobstructed bi-directional migration of molecular species between the enclosed cells and outside environment. Pancreatic cells or islets can be introduced into the pocket in two ways, by puncturing one of the hydrogel sheets of the already formed and sealed pouch using a syringe needle, catheter, or tubing so that the created puncture hole is used to deliver and place at the desired locations, in the volume desired, using a syringe needle, catheter, or tubing.

Once the pancreatic cells are loaded into the inner space of the BAP, the loading port needs to be sealed. One can use a procedure similar to that described for sealing the edges of the BAP pouch that is using dissolution of PVA hydrogel at the loading site. As mentioned earlier this is a process of localized dissolution of hydrogel sheet rather than a melting process because of solvent being present.

Thus, one can do the following: If a puncture hole in the hydrogel sheet is small like one made by a syringe needle gauge 20 or smaller, the entry port can be left untreated, since the hydrogel is self-sealing, the puncture hole will seal and prevent leakage or to ensure that no potential pathway is available for cells of the immune system to penetrate the immuno-isolation space of the BAP pouch, the site where the sheet has been punctured will be sealed using one of possible methods as described Supra dealing with sealing the edges of the pouch.

The preferred and simple method is to use a steam nozzle to soften or dissolve the site of puncture and create a new hydrogel that completely seals and erases the puncture site using a warm to hot object like a metal spatula and then touching the site where the puncture is made. It will seal dissolve and seal the puncture opening or be warmed by a hot object like a metal spatula by touching the site where the puncture was made, and it will dissolve and seal the puncture or use a hot PVA solution such as at 80 to 90° C. and letting one drop fall to the puncture hole. It will seal by dissolving the sheet and making a new hydrogel.

Sealing of the loading hole is not necessary and does not need to be aesthetically pleasing provided that the entry port is positively sealed either by dissolution of the sheet that contains hole or involves also other sheets. The way that the hole is sealed makes no difference provided that the hole is sealed with a new hydrogel. In one case, pre-made tubing sealed to the sheets of the pouch was used. After loading the islets, the tube was sealed.

### Example 7

This example deals with a method of making double walled small diameter tubing that serves concurrently as BAP and as a vascular graft. The entire double-walled tubular BAP was made from PVA Hydrogels. The resulting BAP is a concentric tubular BAP pouch. The BAP pouch that has concentric tubular configuration of double walled tube serves as vascular graft, which is anastomosed to the blood vessel of the host to provide blood circulation.

The procedure as outlined Example 1 was used to make PVA solution and then: A PVA solution was used to make PVA tubes using molding and extrusion. Tubes made using the extrusion process were rapidly crosslinked by submersion into an IPA bath at −35° C. In this example, the inner and the outer tubes are made from a non-immunogenic PVA hydrogel. It is critical to make sure the inner tube is made from PVA hydrogel of the present invention to assure the following: by using non-immunogenic and non-thrombogenic biomaterial like the hydrogel of the present invention, the inner tube serve as a small diameter vascular graft providing intimate contact of the pancreatic cells in the blood stream.

Non-immunogenic hydrogel biomaterial provides free bi-directional transport of all molecular species across the wall of the inner tube without triggering recognition as a foreign body (FB). It also provides true immuno-isolation for pancreatic cells.

The biocompatibility level of the biomaterial used to make outer tubing is not critical as long as the material does not interfere with homeostasis of pancreatic cells. This is because it serves only as a mechanical barrier preventing molecular and cellular components of the immune system reaching the inner space of the double walled tubular BAP housing pancreatic cells.

In this example the outer tubing is also made from non-immunogenic PVA of the present invention resulting in a concentric tube BAP.

The inner tubing had the following dimensions: ID about 3 mm and the OD about 4 mm and wall thickness about 0.5 mm providing an immuno-isolation free bi-directional transport of molecular species.

The outer tubing had the following dimensions: ID about 5 mm and the OD was about 8 mm and the wall thickness was 1.5 mm serving only as mechanical support for the entire BAP and its immuno-isolation provides no critical functions. Sealing the ends of the double walls tubing was accomplished by cohesive bonding described in Example 5 and 6. Example 6 shows the loading of the pancreatic cells and the sealing of the loading port of the double walled tube that serves as a BAP, that has its own vascular grafts.

### Example 8

This example deals with a method of making double walled small diameter tubing that serves concurrently as a BAP and as a vascular graft. The inner tubing of the double-walled tubular BAP was made from PVA hydrogels of the present invention, and the outer tube was made from polyethylene (PE).

The resulting BAP is a concentric multi-material tubular BAP pouch. The BAP pouch that has a concentric tubular configuration of a double walled tube serves as a vascular graft, which is anastomosed to the blood vessel of the host to provide blood circulation. The same procedure and the same performance parameters as discussed in Example 7 apply equally here.

The outer tubing in this case was made from medical grade polyethylene, which is available commercially. The sealing of a PVA hydrogel that is a hydrophilic biomaterial that contains water and PE (which is hydrophobic polymer) was designed with dual action. Dual sealing of both ends of the double walled BAP in the form of tubing was carried out.

First, the ends of PE tubing were functionalized by submersion into oxidizing media and then PVA hydrogel tubing was sealed using a cyanoacrylate glue. To ensure complete sealing at the ends, the PVA tubing was folded back onto the PVA tubing to increase the surface contact between the PVA and the PE tube and glued using cyanoacrylate glue. Loading pancreatic cells and sealing the loading port of the double walled tube serves as the BAP that has its own vascular grafts is outlined in Example 6.

### Example 9

This example deals with a method of making a BAP pouch, which has imbedded single or multiple tubing passing through inner space of BAP. These tubes serve as vascular grafts that are anastomosed to the blood vessel of the host to provide blood circulation through the BAP. The same procedure as outlined in Example 5 for preparation of the BAP and Example 6 that deal with loading pancreatic cells apply here except for the following.

The object of the present example is to provide a safe solution for as close contact between embedded pancreatic cells and the bloodstream as practically possible. In this example rectangular and oval shaped forms of BAP were made and single and multiple PVA hydrogel tubing serving as vascular grafts were placed so that they passed through the inner space of the BAP.

These vascular grafts have a 2 mm ID. These PVA Hydrogel based vascular grafts are non-immunogenic and non-thrombogenic and as such can serve as small diameter vascular grafts for long term functions of vascular grafts. It is critical that these vascular grafts serve not only as conduits for blood flow, that is as classical vascular grafts, but also must have the nature of the walls such that they provide free bi-directional exchange of molecular species important for the functions and establishment of hemostasis of pancreatic cells or islets, that is, immune-isolated within a BAP inner space.

These vascular grafts can be of any size. In this example 2 mm vascular grafts were used. These grafts can serve as an initial supply of molecular species to islets reducing the need for revascularization or can serve as the only supply route of molecular species without regard to the level of vascularization around the BAP. These PVA tubes serving as vascular grafts are cohesively sealed to the walls of the BAP using the same procedure as outlined in the Example 5 dealing with cohesive bonding of edges of sheets making a BAP pouch and sealing puncture loading ports.

### Example 10

This example deals with a method of making a BAP with vascular grafts attached cohesively to the surface of the BAP. These tubes serve as actual vascular grafts that are anastomosed to the blood vessel of the host to provide blood circulation in intimate contact with the surface of BAP. The same procedure as outlined in Examples 5 and 6 for preparation of the BAP, and Example 6 that deals with loading pancreatic cells sealing puncture loading ports, apply here except for the following: A vascular graft was attached to the outer surface of a BAP pouch using the procedures for formation of cohesive bonding between different PVA hydrogels as discussed in the Example 9.

### Example 11

This example deals with a method of imbedding sutures. Importance of imbedded sutures is in doing anastomosis of small diameter vascular grafts used in making BAP and the immobilization of the BAP to an adjacent tissue of a pocket and to the wall of the blood vessel when the BAP is inserted into a blood vessel. Imbedded sutures in this invention have benefits over classical suturing because they provide tear-free suturing of hydrogels because sutures are imbedded in the hydrogel. This suturing creates leak free anastomosis, and rapid and simple suturing can be had.

One can use classical suturing by penetrating the BAP edge with a suture and tying the suture to the wall of a blood vessel or a tissue at the site of the placement of the BAP construction. There are multiple problems with the use of classical suturing of hydrogels. For instance, when the suture penetrates through the hydrogel it forms the hole, which will be enlarged when the suture is under tension and the suturing will be loose. Also, such an enlarged hole in the hydrogel easily undergoes tears because all hydrogels have a low tear strength.

A more desirable method is a method of suturing using sutures imbedded into hydrogel walls of the BAP housing immune-isolated islets. By using imbedded sutures into a wall of the BAP one will completely eliminate drawbacks of classical suturing. The criterion for the selection of material for sutures that can be imbedded into PVA hydrogels is that the material from which sutures are made must be capable of forming hydrogen bonds with the PVA hydrogel matrix and thus it must be cohesively bonded (not adhesively bonded) to the PVA hydrogel. This means that the suture will never be detached or pulled out of the hydrogel, or de-bonded, unless the hydrogel matrix undergoes tearing and thus full destruction.

Materials that can be used for making such sutures are PVA fibers, silk, cellulose-based fibers, or any other material such as surface-modified PE fibers that are capable of forming hydrogen bonds with the PVA hydrogel matrix. PVA fibers are preferred because of their intrinsic high strength that is higher than any other known man-made fiber, especially if the PVA fibers are ultra-drawn.

### Example 12

This example deals with methods of making a BAP that has on its outer surface, scaffolding, that will guide and accelerate revascularization around the insertion pocket.

This example deals with the construction of scaffolding on the surface of the BAP to initiate and guide the growth of vascular vessels to intimate proximity of the surface of an implanted BAP. In this example, BAP in the form of a two-hydrogel-sheet pouch was made so that it consisted of two segments wherein one segment is the actual BAP pouch consisting of immuno-isolation walls that form the inner space of the pouch. It is made from a PVA hydrogel of the present invention described in Example 5.

This will accelerate revascularization. This is possible because the PVA hydrogels of the present invention provide controlled release of bioactive molecules with the ability to provide temporal and spatial distribution of such release including customized rates of release, sequential and intermittent release made by 3-D printing.

The scaffolding is made either only from a bio-erodible PVA hydrogel or a combination of bio-erodible/biodegradable PVA biomaterials and non-bio-erodible biomaterials (enzymatically stable) made from PVA such as electrospin PVA fibers or cellular matrices, or using other biodegradable biomaterials such as alginate, PLA, GLA, PEG, etc. all of them combined into a composite scaffolding placed on the outside surfaces of the BAP.

When PVA is used to make bio-erodible biomaterials one can use low molecular weights fully hydrolyzed or partially hydrolyzed PVA polymer. Molecular weights as low as 5,000 and as high as 50,000 can be used and combined in 3-D structures that will have special and temporal distribution. The rate of bio-erosion is controlled by the selection of desired low molecular weight fully hydrolyzed and partially hydrolyzed PVA polymer or by a combination of PVA with sugar, starch, or other biomaterials that can provide bio-erodible and biodegradable biomaterials that can serve as scaffolding for blood vessel growth. Also, bulk PVA hydrogels in combination with other biopolymers as mentioned Supra or in combination with cellular PVA Hydrogels can be used to make scaffolding.

Pancreatic cells can be loaded into the pouch of this composite BAP either (a) before insertion of the BAP into a surgically made pocket in a tissue or (b) BAP can be loaded after full revascularization is reached. The preferred route is the later one because it will cause a lesser, if any, loss of vitality of the loaded pancreatic cells due to exposure of the cells to hypoxia that is likely to take place in the former case.

### Example 13

This example deals with methods of making Extracellular Matrix (ECM) for enclosed pancreatic cells within the inner space of BAP

### Use of Native Acellular Pancreatic Matrix

In one example islets are loaded into BAP pouch together with their native ECM tissue, that is, use of a homologous acellular pancreatic matrix as a scaffold for islet culture to reside within BAP pouch or being imbedded into PVA hydrogel. Such a transfer is expected to increase the ability of islets to maintain their long-term viability and function. Thus, this aids in long-term survivability of islets. Decellularized xeno-tissue like porcine pancreatic ECM can be used. This is a possibility because ECM will be immune-isolated from the immune system of the host.

An ECM microenvironment for islets was made by placing untreated a fibrous network made by electrospinning PVA fibers having dimensions 0.8 to 2 microns which mimic natural ECM fibrosus network, mixing the electrospun PVA fibers with collagen fibers to minimize the need for modification of PVA fibers.

Treating the PVA fiber network with the components of the actual ECM such as glycoproteins and GAGs (Glycosaminoglycan (GAG), a polysaccharide, to provide proper types of epitopes (functional groups) to aid islets in undergoing binding.

The use of cellular PVA hydrogels with controlled water content of the cell walls, of open cell sponges, serves as the ECM. One makes open cell sponges using preforming methods that use pore forming materials that need to be removed prior to placement into the BAP, such as salt particles. A method of making open cell sponges under this invention does not require any need to remove the particles by washing. Such materials can be proteins, sugar, starch, or similar materials that can remain within the porous structure and become metabolized.

One can treat the PVA fiber network and the walls of the open cells of the reticulated foam with the components of actual ECM's such as glycoproteins and GAGs (Glycosaminoglycan (GAG), a polysaccharide, to provide proper types of epitopes (functional groups) to aid islets to undergo binding.

It is utmost critical to make sure that the BAP that houses islets has the following characteristics: completely immune-isolates islets without activating molecular and cellular effectors of the immune system to prevent triggering foreign body (fb) responses and connective tissue deposition:

Provide free bi-directional transport of all molecular species critical for establishment of islet homeostasis (state analog to the native islet environment) to assure BAP provides a functional cure for diabetes.

Do not disturb laminar flow of blood to prevent triggering onset of clotting cascade and thus the formation of thrombi at the site of placement of the BAP, and downstream, and assure that the BAP device can be easily and safely retrieved in its entirety from the blood stream especially if placed into a portal vein or similar blood vessels.

The following examples serve as general examples of designs of BAP implantable into a blood stream that satisfy all of the above conditions.

### Example 14

This example deals with a method of making a BAP in the form of hollow treads that house and immune-isolated islets. PVA solutions made according to the procedures described in Example 2 and Example 3 is used with the standard manufacturing extrusion procedure to make hollow treads having dimensions of: ID 1 mm, OD 2 mm and wall thickness 0.5 mm. The length of actual implantable BAP is determined by the number of islets incorporated within the hollow tread, which can range from dozens of inches to several feet. These treads were filled with islets or pancreatic cells using 19 gage needles attached to a syringe barrel. The ends of the hollow treads we sealed using procedures described in Examples 5 and Example 6.

The BAP made in this example are intended for placement into a portal vein, though they can be placed into other blood vessels or even into a pocket made in a tissue that has well developed vascular plexus (high density of blood vessels) or pocket that has been pre-vascularized. These BAP are immobilized by suturing to the wall of the blood vessels to prevent uncontrolled migration of the BAP within the vasculature.

### Example 15

This example deals with a method of making a BAP in the form of solid (non-hollow) fibers or threads, and narrow ribbons or tapes that are made by extrusion of PVA Hydrogel that has imbedded immune-isolated islets. This example follows the same procedures as described in the Example 14 except that the extrusion used PVA solutions made according to the procedure described in the Example 3. One variation to the above process is to use PVA solution prepared according to Example 4, that is to make fibers, treads, ribbons and tapes that are made in the form of cellular PVA that houses imbedded and immune-protected islets within the walls of an open cell sponge.

### Example 16

This example deals with methods of making tubular BAP's containing immune-isolated islets. This example uses procedures that are similar to those described in Example 15 except for the following: hollow round (circular) tube or oval shaped tube or ribbon shaped tube is made by extrusion as described in the Example 15 using bulk PVA Hydrogels. The length of these tubular BAP are much shorter than those BAP described in Example 15.

Typically, they are 6 inches to a foot long, depending on the intention of having a predetermined number of immune-isolated islets within the BAP. Round tubes in this case have dimensions of: ID 2.5 mm, OD 3.5 mm and wall thickness of 0.5 μm.

A slight variation to this example is to use 6 μm PVA fibers for braiding and thus providing reinforcement. Only 5% by volume PVA fibers were used as compared to the volume of PVA hydrogel matrix to provide low level of braiding and thus minimal interference with free bi-directional migration of molecular species through the wall of the tubular BAP.

### Example 17

This example deals with a method of making catenated beads that encapsulate pancreatic cells within sold or hollow beads.

Example 23 describes a process for making catenated beads that immune-isolate islets. The connection, i.e., catenation, of encapsulating beads is accomplished by pre-treating surface of the 6 μm PVA fibers with a dilute hydrogel solution to assure bonding to the beads and to assure non-immunogenic response of fibers.

A slight variation of the above-described example of 1-D catenated BAP is to make 2-D catenated beads of BAP. This 2-D form of catenated BAP can be placed into portal vein but can also be placed into highly vascularized tissue.

It is important to design BAPs that have a high surface area to volume (S/V) ratio. This is because one must provide the maximum possible flux of bi-directional transport of molecular species important for maintaining normal functions of islets and thus for islets to establish a needed level of homeostasis. Free bi-directional migration of the following molecular species is critical for islet functions which can be oxygen, carbon dioxide, glucose, insulin, and other hormones.

All of the contemplated structures can be made by classical processing such as extrusion, molding, casting or using 3-D printing since PVA solutions are ideally suited for 3-D printing. Also, these BAP can be made using 3-D tissue biopriming because PVA hydrogels of the present invention require the use of mild conditions for manufacturing of tissues that do not affect the well-being and functions of pancreatic cells. See FIG. 27 through FIG. 29.

### Example 18

This example deals with methods of making BAP pouches that have multiple holes to increase the surface/volume ratio of the BAP.

This example uses BAPs made according to the Example 5 that have been filled with islets to make functioning BAP pouches. In this case the surface/volume (s/v) ratio was increased by simply punching holes through the BAP pouch using heated hole punching tool (i.e. tubular toll similar to the one used to make holes in the leather). By using a heated tool, one creates perforations and at the same time dissolves both sheets of BAP made in the form of a double sheet pouch and dissolution of the edges of the hole leads to cohesive sealing of edges of each hole. These holes allow for the flow of the surrounding fluid through them and allow blood vessel to grow through them during angiogenesis of vascular plexus, which provides increased level of intimate contact between blood vessels and the surfaces of BAP.

Another variation is to use a single sheet BAP, that is, use of a BAP made from hydrogel imbedded islets and then punch holes. Sealing of the edges of the holes is critical. By making holes in this case one loses some islets but gains in S/V ratio.

### Example 19

This example deals with a method of making single sheet BAP's consisting of imbedded islets in the form of a honeycomb with a surface/volume ratio of BAP. BAP was made in the form of honeycombs from PVA solutions based on glycerin that has imbedded islets or pancreatic cells. The BAP was made using a mold that results in multiple honeycomb openings. This procedure of making BAP from a single PVA hydrogel sheet that houses immune-isolated islets allows a major increase in the s/v ratio. Different variations of this concept are possible.

### Example 20

This example deals with a method of making a BAP that has a corrugated surface to increase the surface/volume ratio of the BAP. This BAP has an enhanced s/v ratio through the use of corrugated PVA hydrogel sheets that can be sandwiched between two flat PVA hydrogel sheets or simply cohesively bonded to another floating PVA Hydrogel sheet. Corrugation naturally creates channels.

In order to increase the s/v ratio, each consecutive channel formed by the corrugated sheet will have spaces for filling with pancreatic cells. After cohesively sealing the ends of each channel using the procedures described in the Example 5, each channel provides immuno-isolation of pouches for pancreatic cells, with increased surface area due to corrugation surface.

These structures were made using extrusion, casting and molding PVA hydrogels of the present invention. Islets are introduced into channels using channel end openings of each corrugation and sealed as described in Example 5, 6, 7 and 8. Corrugated structures were made using pouch type designs of BAP and required corrugation of one sheet cohesively bonded to the other sheet that stays flat. This structure created channel-like spaces that were filled with islets and sealed cohesively by bonding the ends of the channels.

In another example, single corrugated sheets that contain imbedding islets was used. The resulting structure is similar to the one described in Example 20 but contains no free space, only single sheet corrugation that contains imbedded islets.

### Example 22

This example deals with methods of making extruded comb-like structures that increase surface/volume ratios of BAP. One can use any PVA hydrogel described in Examples 1, 2, 3 and 4. Comb-like BAPs possess very large s/v ratios. They can be made using extrusion, casting and molding. One has an advantage of dealing with only one sheet that houses imbedded islets within the PVA Hydrogels. Comb-like structures can be placed on one surface or on both surfaces (front and back) of the BAP. This example is made with continuous comb-like structures that are like multiple ribbons glued to the surface. However, one can use any shapes of protrusions including rods, gratings, etc. that will lead to an increase of surface area.

### Example 23

This example deals with a method of making 2-D catenated encapsulating solid beads that increase the surface/volume ratio of the BAP. One can increase the s/v ratio if one encapsulates islets into small beads like 0.5 and 1.0 mm radius. The beads are interconnected (catenated) using pretreated PVA fibers into a string, that is, 1-D structure or into 2-D structure. This catenation of beads increase the s/v ratio.

Beads containing islets are connected using 6 μm PVA fibers that have been surface pre-treated. Surface treatment of the PVA fibers was done by immersion the PVA fibers into a PVA solution, made using mixed solvents and glycerin, and kept at 65° C. Duration of immersion of fiber was 3 seconds.

The surface treatment of PVA fibers was necessary for two reasons (1) to soften surface of fiber to provide cohesive bonding to the PVA beads that immune-isolate islets and (2) to make the surface of fibers non-immunogenic and non-thrombogenic because solid, crystalline PVA is known to be immunogenic and thrombogenic.

There are two possible locations into which immunoisolated islets can be placed in a body of a host to perform as a functional cure for diabetes. One is the direct placement of immunoisolated islets into a blood vessel. Placement into a blood vessel is the most desirable location since such placement inherently would meet practically all of the pre-requisites the immunoisolation capsule must meet. However, this route faces a set of the most difficult biomaterial-related requirements that must be met concurrently. No material was found in the prior art that could meet such stringent requirements, especially the unforgiving requirements of hemocompatibility that has placed this route on hold for over 20 years.

The second method is the placement of a Bioartificial Pancreas (BAP) pouch into a surgically created pocket placed subcutaneously or intraperitoneally or in a muscle, liver or other tissue that is capable of providing a well-developed vascular plexus and can provide sufficient and timely bi-directional migration of all molecular and gaseous species.

The instant invention relates to the development and placement of a BAP containing immunoisolated islets into a surgically prepared pocket in a tissue. Such a placement of the BAP placed into a tissue, faces two independent sets of challenges. One set of challenges is obviously related to the design of the BAP itself that can concurrently provide immunoisolation of islets, fibrosis-free surfaces and free bi-directional migration of all molecular species and the second challenge is related to the hurdles imposed by the placement itself into an insertion pocket created surgically within a tissue.

There are two key issues in this case, the need for pre-vascularization and complete healing of the insertion pocket prior to insertion of the BAP. These issues are resolved using a PVA hydrogel of the present invention, that is, non-immunogenic properties that can provide a full control of a release of bioactive molecules.

In order for the immunoisolated islets inside a pouch or tube of BAP to provide a functional cure for T1D, one must ensure that the exchange of oxygen, nutrients, hormones, glucose and other molecular species is essentially unobstructed. Any significant restriction may lead, at least, to undesirable islets delayed response to insulin releases due to changes in glucose level in the blood stream, or islets may adopt a quiescent state by functioning only at the minimum cell-life support level. These may not be severe changes, but they still make a BAP not fully functional. If the BAP requirements are not met, it is highly likely that islets will undergo dedifferentiation most likely into non-contributing cells or undergo reduction in islet population or even undergo apoptosis due to starvation and suffocation. The requirements that must be met in order for an implanted BAP to serve as a functional cure for T1D are strict and exacting and must be fully met since a partial meeting of requirements is unacceptable and will not lead to a functional cure of T1D.

Therefore, one must assure that the exchange of molecular species between the blood stream and the immunoisolated islets is timely and fully unobstructed, that is, exchanges of all molecular species are taking place at the diffusion flux equal or higher than the required level for maintenance of cells homeostasis, that is, at the same level as the islets experience in their native environment.

Before a BAP made in the form of pouch-like device that houses islets in accordance to the instant invention can be placed into a host's tissue, one must surgically create a place typically called a pocket for insertion of the BAP. Insertion of the BAP into such a tissue pocket faces some serious issue related to survival and ability to maintain vital islets which must be resolved prior to insertion of the artificial BAP of this invention that houses immunoisolated islets. Literature data show that if the surgical pocket issues are not resolved, it is likely that a significant portion of islet population within the BAP will be lost.

The instant invention deals with the use of a device such as a sheet made from non-immunogenic hydrogels of the present invention that concurrently resolve the issues inherent to the surgically created insertion pocket that islets must deal with. In fact, there are two independently caused sets of issues that must be resolved prior to insertion of the actual BAP into the tissue pocket in order for the inserted BAP to provide a functional cure for diabetes.

One such issue is that one must generate a well-developed vasculature (pre-vascularization) in as close proximity to the surfaces of the BAP as practically possible prior to insertion of the BAP. If this is not accomplished, then a vast majority of islets will undergo apoptosis due to suffocation caused by the lack of oxygen (hypoxia) and due to starvation caused by the lack of access to nutrients and if some islets somehow manage to survive the unfriendly environment, the islets will not be able to respond with a hormone release to regulate glucose level in a blood stem.

The literature data indicates that if insufficient vascularization is not accomplished prior to insertion of BAP, one may experience a loss of up to 95% of the islet population housed within a BAP.

One must initiate an accelerated wound healing that will also lead to full control of an inflammatory response of the injured tissue during the surgical creation of an insertion pocket in a tissue. If accelerated wound healing is not initiated, acute inflammatory activity will remain high with a tendency to advance to a chronic inflammatory response which will result in a build-up of a significant population of pro-inflammatory M1 phenotype macrophages that will create a very unfriendly environment for the islets. This is because highly active macrophages, especially the M1 phenotype, will release cytokines which in combination with immunoglobulin and a complement system will lead to a destruction of islets through apoptosis (programmed death). This is serious, and a real threat to islets and the appearance of such a threat should not be a surprise to anybody understanding immunology.

High M1 activity will aid in the progression of inflammation from an acute to a chronic inflammatory response that can lead to a potentially fatal cytokine storm that may be very harmful to the host itself.

All these phenomenon related to molecular effectors of immune system are happening because of the fact that even the best-designed BAP will provide immunoisolation to islets only from the cellular effectors of an immune system but would leave islets completely exposed to all molecular effectors of the immune system. This threat is inevitable because when one is designing BAP immunoisolation, one must allow free bi-directional migration of molecular species through the immunoisolation membrane of the BAP. This means that all molecular effectors of the immune system can also freely reach the islets.

By reducing the pro-inflammatory phenotype population of macrophages, and promoting the increase of M2 pro-healing or pro-resolution macrophage phenotype population, wound healing will accelerate

Using PVA hydrogels of the present invention, one can mitigate and practically eliminate the threat to the enclosed islets from cytokines and complement system peptides, aided by immunoglobulin and other opsonizing molecules. Typically, these molecular components of the immune system will try first individually to kill opsonized (labeled) cells as foreign bodies but then if they fail doing it individually, they will join forces to kill the islets together. This is serious threat to viability of islets because in certain human populations that threat will lead to death of the entire islet population.

These two sets of issues that are inherent to the surgical creation of the insertion pocket for the BAP of this invention must be resolved prior to insertion of the BAP into the pre-created pocket in a tissue. The resolution to these issues is best served using a method based on non-immunogenic and controlled drug release PVA hydrogels of the present invention. This method can, in a pretty simple manner, be very effective in solving the issue of minimizing, and likely completely eliminating, all hurdles inherent to a surgically created insertion pocket for the BAP.

As cytokines have a central role in establishing the balance between pro- and anti-inflammatory immunity, their manipulation in chronic disease has been, and remains, a core interest for development of therapeutic interventions that seek to mitigate the consequences of non-resolving inflammation. TNF-α is a strong, non-resolving pro-inflammatory cytokine and it seems that TNF is at the apex of a pro-inflammatory cascade together with IL-1β that likely has the same type of receptors for blocking their cell activity. Inhibiting TNF reduces inflammation by interrupting non-resolving inflammation. Anti-TNF drugs were thereafter used in millions of patients with chronic inflammation.

By blocking TNF activity, one promotes an increase in M2 populations of polarized macrophages that will lead to a completion of wound healing and to homeostasis of the tissue. There is strong interaction on different levels between TNF's inhibitory activity on the M2 macrophage population.

Generation of proinflammatory macrophages of M1 phenotype is the result of stimulation with IFNγ, a pro-inflammatory cytokine and lipopolysaccharide (LPS) and is associated with a Th1 environment. IFNγ I coordinating the innate and adaptive immune response M1 macrophages are producing IL-12, IL-6, TNF and iNOS. M2 macrophage generation results from stimulation with IL-4 or IL-13 and is associated with a Th2 environment.

Immunoglobulin (Ig) is an antibody, an adaptive immune system effector molecule that has two roles in immune protection of body wherein the main role of Ig is opsonization/tagging of non-self-cells such as would be the case with islets within the BAP for destruction by other effectors of immune system and one can neutralize directly opsonized cells by, for example, blocking essential pathways.

Immunoglobulin work with the complement system in the earliest phases of an immune response to help facilitate clearance of the offending antigen and delivery of the resulting immune complexes to the lymph nodes or spleen using adaptive immune system cellular effectors. Ig's tagging and working together with cytokines, complement system, TNF, etc. will affect islets enclosed in BAP. Thus, the adaptive system has its own opsonization molecules such as immunoglobulin antibodies that have a primary role to tag the invader's non-self surfaces.

A complement system has the ability of destroying non-self-cells on its own by destroying the membrane of the non-self-cell. This complement system mediated cell death requires the cooperation of many large molecules acting in concert, which could lead to destruction of islets in BAP. Thus, to control inflammatory activity one needs to control macrophage activity using anti-inflammatory drugs.

There are two main reasons why the PVA hydrogels of the present invention are the most desirable choices of biomaterials to resolve the issues related to the insertion pocket of the BAP in a tissue.

PVA hydrogels of the present invention are inherently non-immunogenic, which means that these biomaterials will not activate the immune system. This means that no foreign body recognition will take place and also as a consequence, these biomaterials will not adhere to adjacent tissue. This is a very important fact because, after a certain length of time, such as typically 2 to 3 weeks, inserted hydrogel objects (like a sheet) will be removed from a fully healed and re-vascularized pocket but without tearing adjacent tissue.

Tearing will not take place because the tissue cannot adhere to the surfaces of these hydrogels. This faithfully preserves the shape and size of the insertion pocket. Thus, one of the most critical characteristic of the PVA hydrogels of the present invention is the absence of tissue adhesion to the surfaces of the inserted hydrogel object. Removal of an insert does not require additional surgery to cut adhered tissue from hydrogel insert that would otherwise generate a high level of inflammatory response and thus defeat the whole purpose of using hydrogel insertion device.

The second reason is that the PVA hydrogel of the present invention is inherently one of the best matrices for controlled drug release including burst release. All of the modes of release are critical for accomplishing the two goals namely, create pre-vascularized plexus in as close proximity to surfaces of inserted BAP as possible. This is accomplished by releasing in a controlled predetermined manner bioactive molecules such as Angiopoietin-2, Fibroblast Growth Factor (FGF) (Promotes proliferation & differentiation of endothelial cells, smooth muscle cells, and fibroblast), Vascular Endothelial Growth Factor (VEGF-A), Platelets Derived Growth Factor (PDGF), Fibroblast growth factor (FGF) important in de novo organization of EC in vasculogenesis and transforming TGF (TGF-1β). These growth factors, cytokines, chemokines etc. will initiate and accelerate angiogenesis for formation of vasculature in the proximity of BAP surfaces. This pre-vascularization must be accomplished before BAP insertion to avoid loss of islets.

One must completely heal the injured tissue that resulted during creation of the insertion pocket. The wound healing process will inevitably lead to a reduction of the inflammatory activity and prevention of acute inflammation progressing to chronic inflammation. Initiation and acceleration of wound healing and prevention of chronic inflammation development is accomplished by releasing, in a controlled predetermined manner, bioactive molecules such as antimicrobial, growth factors, cytokines and chemokines. Complete healing of the tissue will minimize and practically eliminate potentially detrimental activity of cytokines and complement proteins that will likely lead to the death of the islet population within the BAP. The level of inflammatory response must be under full control before the BAP housing the islets is inserted into the surgically prepared pocket in a tissue. It is particularly important to heal the wound and bring inflammatory activity to an acute level of activity preferentially to the low level that corresponds to daily maintenance level.

Therefore, before actual BAP housing immunoisolated islets are inserted into the surgically created pocket in a tissue, one must concurrently initiate accelerated (a) wound healing together with controlling inflammatory response of the wounded insertion pocket and (b) pre-vascularize both walls of the insertion pocket surgically created within a tissue.

After the pocket has been surgically created within a tissue, non-immunogenic sheet made from PVA Hydrogel of the present invention that has identical dimensions to the actual BAP pouch is first preloaded with growth factors, cytokines, chemokines and anti-inflammatory bioactive molecules and then inserted into the surgically created pocket. After an insertion pocket in a tissue has been healed and pre-vascularized, the BAP housing the immunoisolated islets is inserted into the pocket and the BAP immobilized using pre imbedded sutures.

What is claimed is:

1. A physically crosslinked polyvinyl alcohol hydrogel prepared using mixed solvents, containing continuous, uninterrupted immobilized water, said hydrogels having at least 88% by weight water content or higher and a syndiotacticity of fifty percent or greater.

2. The physically crosslinked polyvinyl alcohol hydrogel as claimed in claim 1 wherein said hydrogel has at least 88% by weight water content or higher.

3. The physically crosslinked polyvinyl alcohol hydrogel as claimed in claim 1 wherein said hydrogel is reinforced using materials selected essentially from the materials selected from the group consisting of:
a) long fibers,
b) woven fibers,
c) woven fabrics,
d) non-woven fibers,
e) non-woven fabrics, and
f) combinations of a) to e).

4. The physically crosslinked polyvinyl alcohol hydrogel as claimed in claim 1 wherein said hydrogel contains less than 1% acetate groups.

5. The physically crosslinked polyvinyl alcohol hydrogel as claimed in claim 1 wherein said low temperature is in the range of from −35° C. to 40° C.

6. The physically crosslinked polyvinyl alcohol hydrogel as claimed in claim 1 wherein said hydrogel is prepared using a low viscosity.

7. The physically crosslinked polyvinyl alcohol hydrogel as claimed in claim 7 wherein the viscosity is in the range of 0.2 Pas or higher at 25° C.

8. The physically crosslinked polyvinyl alcohol hydrogel as claimed in claim 1 wherein said hydrogel is formed using 3-D printing.

9. The physically crosslinked polyvinyl alcohol hydrogel as claimed in claim 1 wherein said mixed solvents are selected from the group consisting of:
i) water, ii) dimethylsulfoxide, iii) glycerin, iv) ethyl alcohol, v) propanol, vi) butanediol, vii) poly(ethylene glycol), viii) propylene glycol, ix) poly(propylene glycol), x) tri-ethylene glycol, and combinations of i) to x) in the range of 2 weight percent water to 98 percent water.

10. A physiologically and mechanically biocompatible artificial pancreas, said pancreas comprising a combination of the hydrogel said hydrogel being a physically crosslinked polyvinyl alcohol hydrogel prepared using mixed solvents, containing continuous, uninterrupted immobilized water, said hydrogels having at least 88% by weight water content or higher and a syndiotacticity of fifty percent or greater.
combined with cells, said cells being selected from the group consisting of islets; clusters of islets; Beta cells, and combinations of islets and Beta cells.

11. The physiologically and mechanically biocompatible artificial pancreas as claimed in claim 10 wherein said physiologically and mechanically biocompatible artificial pancreas is placed directly in a blood vessel.

12. The physiologically and mechanically biocompatible artificial pancreas as claimed in claim 10 wherein said pancreas contains vascular grafts thereon or therein.

13. The physiologically and mechanically biocompatible artificial pancreas as claimed in claim 12 wherein said vascular grafts are manufactured from said hydrogels as claimed in claim 1.

14. The physiologically and mechanically biocompatible artificial pancreas as claimed in claim 10 wherein said pancreas contains vascular grafts therein.

15. The physiologically and mechanically biocompatible artificial pancreas as claimed in claim 10 wherein said pancreas is created by encapsulating said cells in said hydrogel.

16. The physiologically and mechanically biocompatible artificial pancreas as claimed in claim 10 wherein said pancreas is manufactured as a composite.

17. The physiologically and mechanically biocompatible artificial pancreas as claimed in claim 10 wherein said physiologically and mechanically biocompatible artificial pancreas has sutures associated therewith, said sutures being cohesively bonded to said hydrogel using hydrogen bonding.

18. The physiologically and mechanically biocompatible artificial pancreas as claimed in claim 10 wherein said physiologically and mechanically biocompatible artificial pancreas is formed into a configuration selected from the group consisting of: a single sheet, double sheets in the form of a sealed pouch, ribbons, hollow ribbons, tapes, hollow tapes, fibers, hollow fibers, threads, hollow threads, rods, hollow rods, tubes, corrugated structures, comb-like structures, ribbed structures, array structures, multiple hole punctured structures, micro-capsules, capsules catenated in 1-D configuration, capsules catenated in 2-D configuration, coatings, and combinations thereof.

19. The physically crosslinked polyvinyl alcohol hydrogel as claimed in claim 1 wherein said hydrogel is formed into a pouch manufactured from a hydrogel selected from the group consisting of: sheets, corrugated sheets, multiple hole punctured surfaces, ribbons, tapes, hollow fibers, hollow threads, hollow rods, and hollow tubes.

20. The physically crosslinked polyvinyl alcohol hydrogel as claimed in claim 1 wherein said hydrogel is formed using 3-D printing.

21. The physically crosslinked polyvinyl alcohol hydrogel as claimed in claim 1 wherein said hydrogel is placed directly in a blood vessel.

22. The physically crosslinked polyvinyl alcohol hydrogel as claimed in claim 1 wherein said hydrogel is in the form of a string of catenated beads.

23. The physically crosslinked polyvinyl alcohol hydrogel as claimed in claim 22 wherein said hydrogel is in the form of micro beads.

24. The physically crosslinked polyvinyl alcohol hydrogel as claimed in claim 22 wherein said hydrogel is in the form of macro beads.

25. The physically crosslinked polyvinyl alcohol hydrogel as claimed in claim 1 that is in the form of a corrugated sheet.

26. The physically crosslinked polyvinyl alcohol water-free solid prepared using mixed solvents, said solid being essentially free of water and having a syndiotacticity of fifty percent or greater said solid treated to induce biocompatibility, said treatments selected from the group consisting of:
a) a surface treatment and,
b) bored perforations in said solid.

27. The physically crosslinked polyvinyl alcohol hydrogel as claimed in claim 26 wherein said hydrogel is formed using 3-D printing.

28. The physically crosslinked polyvinyl alcohol hydrogel as claimed in claim 26 wherein said hydrogel is placed directly in a blood vessel.

29. The physically crosslinked polyvinyl alcohol hydrogel as claimed in claim 26 wherein said hydrogel is in the form of a string of catenated beads.

30. The physically crosslinked polyvinyl alcohol hydrogel as claimed in claim 26 wherein said hydrogel is in the form of a string of micro beads.

31. The physically crosslinked polyvinyl alcohol hydrogel as claimed in claim 26 wherein said hydrogel is in the form of a string of macro beads.

32. The physically crosslinked polyvinyl alcohol hydrogel as claimed in claim 1 that is in the form of a corrugated sheet.

33. The physically crosslinked polyvinyl alcohol hydrogel as claimed in claim 26 wherein said hydrogel contains less than 1% acetate groups.

34. The physically crosslinked polyvinyl alcohol water-free solid as claimed in claim 26 containing 1% or less of water.

35. The physically crosslinked polyvinyl alcohol water-free solid as claimed in claim 26 wherein said solid is prepared at a low temperature.

36. The physically crosslinked polyvinyl alcohol water-free solid as claimed in claim 26 wherein the low temperature is in the range of from −35° C. to 40° C.

37. The physically crosslinked polyvinyl alcohol hydrogel as claimed in claim 26 wherein said water-free solid is prepared using a low viscosity.

38. The physically crosslinked polyvinyl alcohol water-free solid as claimed in claim 37 wherein said solid is prepared using a low viscosity in the range of 200 cps to 1,000,000 cps.

39. The physically crosslinked polyvinyl alcohol water-free solid as claimed in claim 26 wherein said mixed solvents are selected from the group consisting of
water, dimethylsulfoxide, glycerin, ethyl alcohol, propanol, butanediol, poly(ethylene glycol), propylene glycol, poly(propylene glycol), tri-ethylene glycol, mixed with water in the range of 2 weight percent water to 98 weight percent water.

40. The physically crosslinked polyvinyl alcohol water-free solid as claimed in claim 26 wherein said solid is hydrated with water after manufacture.

41. The physically crosslinked polyvinyl alcohol water-free solid as claimed in claim 26 wherein said solid is surface treated with a dilute hydrosol solution.

42. A physiologically and mechanically biocompatible artificial pancreas, said pancreas comprising a combination of said water-free solid as claimed in claim 26 and cells, said cells being selected from the group consisting of
i) islets;
ii) clusters of islets;
iii) Beta cells, and
iv) combinations of islets and Beta cells.

43. The physiologically and mechanically biocompatible artificial pancreas as claimed in claim 42 wherein said pancreas contains vascular grafts thereon.

44. The physiologically and mechanically biocompatible artificial pancreas as claimed in claim 42 wherein said physiologically and mechanically biocompatible artificial pancreas is placed directly in a blood vessel.

45. The physiologically and mechanically biocompatible artificial pancreas as claimed in claim 42 wherein said pancreas is created by encapsulating said cells in said hydrogel.

46. The physiologically and mechanically biocompatible artificial pancreas as claimed in claim 42 wherein said pancreas is manufactured as a composite.

47. A method of providing a physiologically and mechanically biocompatible artificial pancreas, said method comprising:
A) providing a physically crosslinked polyvinyl alcohol hydrogel prepared using mixed solvents, containing continuous, uninterrupted immobilized water, said hydrogel having at least 88% by weight water content or higher and a syndiotacticity of fifty percent or greater;
B) treating said hydrogel with cells selected from the group consisting of
i) islets;
ii) clusters of islets;
ii) Beta cells, and
iii) combinations of islets and Beta cells;
C) attaching at least one suture to said physiologically and mechanically biocompatible artificial pancreas.

48. The method of providing a physiologically and mechanically biocompatible artificial pancreas as claimed in claim 47 wherein said pancreas contains vascular grafts thereon or therein.

49. A method of providing a physiologically and mechanically biocompatible artificial pancreas, said method comprising:
A) providing a physically crosslinked polyvinyl alcohol hydrogel prepared using mixed solvents, containing continuous, uninterrupted immobilized water, said hydrogel having at least 88% by weight water content or higher and a syndiotacticity of fifty percent or greater;
B) treating said hydrogel with cells selected from the group consisting of
i) islets;
ii) clusters of islets;
iii) Beta cells, and
iv) combinations of islets and Beta cells;
C) forming a pouch of said physically crosslinked polyvinyl alcohol hydrogel;
D) sealing the edges of said pouch;
E) attaching at least one suture to said physiologically and mechanically biocompatible artificial pancreas.

50. A method of providing a physiologically and mechanically biocompatible artificial pancreas, said method comprising:
A) providing a physically crosslinked polyvinyl alcohol hydrogel prepared using mixed solvents, containing continuous, uninterrupted immobilized water, said hydrogel having at least 88% by weight water content or higher and a syndiotacticity of fifty percent or greater;
B) treating said hydrogel with cells selected from the group consisting of
i) islets;
ii) clusters of islets;
iii) Beta cells, and
iv) combinations of islets and Beta cells;
C) inserting said physically crosslinked polyvinyl alcohol hydrogel into a blood vessel;
D) anastomosing said blood vessel to a part of vascularization of said hydrogel;
E) attaching at least one suture to said physiologically and mechanically biocompatible artificial pancreas.

51. The method of providing a physiologically and mechanically biocompatible artificial pancreas as claimed in claim 49 wherein said pancreas contains vascular grafts thereon or therein.

52. A method of providing a physiologically and mechanically biocompatible artificial pancreas, said method comprising:
A) providing a physically crosslinked polyvinyl alcohol water-free solid prepared using mixed solvents, said solid being essentially free of water and having a syndiotacticity of fifty percent or greater;
B) treating said solid with cells selected from the group consisting of
i) islets;
ii) clusters of islets;
iii) Beta cells, and
iv) combinations of islets and Beta cells;
C) attaching at least one suture to said physiologically and mechanically biocompatible artificial pancreas.

53. A method of providing a physiologically and mechanically biocompatible artificial pancreas, said method comprising:
A) providing a physically crosslinked polyvinyl alcohol water-free solid prepared using mixed solvents, said solid being essentially free of water and having a syndiotacticity of fifty percent or greater;
B) treating said hydrogel with cells selected from the group consisting of
i) islets;
ii) clusters of islets;
iii) Beta cells, and
iv) combinations of islets and Beta cells;
C) forming a pouch of said physically crosslinked polyvinyl alcohol hydrogel;
D) sealing said pouch along said pouch edges;
E) attaching at least one suture to said physiologically and mechanically biocompatible artificial pancreas.

54. The method of providing a physiologically and mechanically biocompatible artificial pancreas as claimed in claim 53 wherein the pouch of said bioartificial pancreas contains vascular grafts therein or thereon the wall of said solid so that said grafts are an integral part of the wall of the bioartificial pancreas.

55. A method of providing a physiologically and mechanically biocompatible artificial pancreas, said method comprising:
A) providing a physically crosslinked polyvinyl alcohol water-free solid prepared using mixed solvents, said solid being essentially free of water and having a syndiotacticity of fifty percent or greater;
B) treating said hydrogel with cells selected from the group consisting of
v) islets;
vi) clusters of islets;
vii) Beta cells, and
viii) combinations of islets and Beta cells;
C) inserting said treated hydrogel into a blood vessel;
D) anastomosing said blood vessel to a part of vascularization of said hydrogel;
E) attaching at least one suture to said physiologically and mechanically biocompatible artificial pancreas.

56. The method of providing a physiologically and mechanically biocompatible artificial pancreas as claimed in claim 55 wherein said pancreas contains vascular grafts thereon or therein.

57. A method of providing a physiologically and mechanically biocompatible artificial pancreas, said method comprising:
A) providing a physically crosslinked polyvinyl alcohol hydrogel prepared using mixed solvents, containing continuous, uninterrupted immobilized water, said hydrogel having at least 88% by weight water content or higher and a syndiotacticity of fifty percent or greater;
B) forming a pouch of said physically crosslinked polyvinyl alcohol hydrogel;
C) inserting cells into the interior of said pouch said cells selected from the group consisting of
i) islets;
ii) clusters of islets;
iii) Beta cells, and
iv) combinations of islets and Beta cells;
D) sealing the edges of said pouch;
E) attaching at least one suture to said physiologically and mechanically biocompatible artificial pancreas.

58. The method of providing a physiologically and mechanically biocompatible artificial pancreas as claimed in claim 57 wherein said pancreas contains vascular grafts thereon or therein.

59. A method of providing a physiologically and mechanically biocompatible artificial pancreas, said method comprising:
A) providing a physically crosslinked polyvinyl alcohol water-free solid prepared using mixed solvents, said solid being essentially free of water and having a syndiotacticity of fifty percent or greater;
B) forming a pouch of said physically crosslinked polyvinyl alcohol hydrogel;
C) treating the interior of said pouch with cells selected from the group consisting of
i) islets;
ii) clusters of islets;
iii) Beta cells, and
iv) combinations of islets and Beta cells;
D) sealing said pouch along said pouch edges;
E) attaching at least one suture to said physiologically and mechanically biocompatible artificial pancreas.

60. The method of providing a physiologically and mechanically biocompatible artificial pancreas as claimed in claim 59 wherein said pancreas contains vascular grafts thereon or therein.

61. A method of pre-vascularization and control of inflammatory activity using a surgically created insertion pocket for a biocompatible artificial pancreas in a living tissue, said method comprising:
A) surgically creating a pocket in living tissue; preloading a hydrogel sheet with a growth factor material and anti-inflammatory bioactive molecules, said hydrogel sheet being a physically crosslinked polyvinyl alcohol hydrogel prepared using mixed solvents, containing continuous, uninterrupted immobilized water, said hydrogels having at least 88% by weight water content or higher and a syndiotacticity of fifty percent or greater;
B) inserting said hydrogel sheet into said pocket;
C) providing a surgical closure to said pocket;
D) causing vascularization through angiogenesis and healing of said live tissue using a controlled and sequential release of substances that promote vascularization of said pocket;
E) causing healing of said live tissue using a controlled and sequential release of wound healing substances into said pocket.

62. A method of placing a biocompatible artificial pancreas in a living tissue, said method comprising:
A) surgically creating a pocket in living tissue;
B) preloading a hydrogel sheet with a growth factor material and anti-inflammatory bioactive molecules, said hydrogel sheet being a physically crosslinked polyvinyl alcohol hydrogel prepared using mixed solvents, containing continuous, uninterrupted immobilized water, said hydrogels having at least 88% by weight water content or higher and a syndiotacticity of fifty percent or greater;
C) inserting said hydrogel sheet into said pocket;
D) providing a surgical closure to said pocket;
E) causing healing of said live tissue using a controlled and sequential release of wound healing substances into said pocket;
F) causing vascularization through angiogenesis of said live tissue using a controlled and sequential release of substances that promote vascularization of said pocket;
G) providing a protocol to activate angiogenesis to form a vascular plexus in said tissue of said pocket;
H) after a predetermined amount of time, surgically removing said hydrogel sheet;
I) inserting a biocompatible artificial pancreas into said pocket;
J) providing a surgical closure to said pocket, said biocompatible artificial pancreas comprising: a physiologically and mechanically biocompatible artificial pancreas, said biocompatible artificial pancreas comprising an immunoisolating enclosure manufactured from said hydrogel combined with cells, said cells being selected from the group consisting of islets; clusters of islets; Beta cells, and combinations of islets and Beta cells.

63. A physically crosslinked polyvinyl alcohol hydrogel prepared using mixed solvents, containing continuous, uninterrupted immobilized water, said hydrogels having a syndiotacticity of fifty percent or greater, a molecular weight of at least 500 to 200,000, a molecular weight distribution of 2.5 or lower, a head-to-tail monomer orientation of 98 percent or higher, total branching of 1 percent or lower, total disordered stereo-tacticity of 70 percent or lower and a preferred degree of hydrolysis of 98 percent or greater.
